(19) United States
(12) Patent Application Publication
(10) Pub. No.: US 2025/0382644 A1
(43) Pub. Date: Dec. 18, 2025

(54) METHOD FOR PRODUCING AMINO ACIDS IN A BIOREACTOR

(71) Applicant: ARKEON GMBH, Tulln an der Donau (AT)

(72) Inventors: Simon Karl-Maria Rasso RITTMANN, Tulln an der Donau (AT); Benjamin SCHUPP, Tulln an der Donau (AT); Barbara REISCHL, Tulln an der Donau (AT); Ruth-Sophie TAUBNER, Tulln an der Donau (AT); Hayk PALABIKYAN, Tulln an der Donau (AT); Christian FINK, Tulln an der Donau (AT); Ipek ERGAL, Tulln an der Donau (AT); Ross FENNESSY, Tulln an der Donau (AT)

(21) Appl. No.: 18/876,312

(22) PCT Filed: Jun. 23, 2023
(86) PCT No.: PCT/EP2023/067080

§ 371 Date: Dec. 18, 2024

(30) Foreign Application Priority Data
Jun. 24, 2022 (EP) 22180983.3

(51) Int. Cl. C12P 13/04; C12M 1/00; C12N 1/20; C12R 1/01

(52) U.S. Cl. CPC C12P 13/04; C12M 21/00; C12N 1/205; C12R 2001/01

(57) ABSTRACT

A method of producing amino acids by fermentation in a bioreactor is disclosed, wherein the bioreactor includes methanogenic microorganisms in a fermentation broth, the method including at least the steps of feeding a gaseous carbon source including carbon dioxide and/or carbon monoxide, a nitrogen source and a sulfur source to the bioreactor under conditions such that the methanogenic microorganisms produce the amino acids; and harvesting at least a portion of the amino acids from the supernatant of the fermentation broth. Preferred methanogenic microorganisms are e.g. archaea selected from Methanothermobacter, Methanothermococcus and Methanococcus. Also provided is a fermentation broth comprising methanogenic microorganisms and amino acids in the supernatant, and a bioreactor including the same.

### TECHNICAL FIELD

The field of present invention relates to methods for producing amino acids by fermentation in a bioreactor with methanogenic microorganisms.

### BACKGROUND

Amino acids are applied in a variety of sectors: food and feed, agriculture, pharmaceuticals and even packaging and housing. The biosynthesis of proteinogenic amino acids is an important branch of biotechnology (Becker and Wittmann 2012). However, the metabolic potential of archaea with regard to amino acid production has up to now been vastly overlooked (Pfeifer et al. 2021). A standardized archaeal taxonomy is disclosed in Rinke et al, 2021.

Methanogenic archaea are known for the ability to generate methane (CH₄) as end product of their energy metabolism (see e.g. Mand & Metcalf, 2019). Some of them are able to grow autotrophic, hydrogenotrophic by reducing carbon dioxide (CO₂) with molecular hydrogen (H₂) to CH₄, and play a crucial role in the global carbon cycle (Lyu et al. 2018). According to their substrate utilization spectrum, methanogens can be divided into different metabolic groups: hydrogenotrophic (H₂, formate or simple alcohols), aceticlastic (acetate), methylotrophic (compounds containing a methyl group), H₂ dependent methylotrophic(methylated compounds with H₂ as electron donor), and methoxydotrophic(methoxylated aromatic compounds) (Mayumi et al. 2016, Kurth et al. 2020). The biology of methanogenic archaea is also discussed in older publications, such as Zeikus, 1977.

Taubner et al., 2019, is an exobiology research paper unrelated to biotechnology. Specifically, the document concerns the membrane lipid composition and amino acid excretion patterns of Methanothermococcus okinawensis grown in the presence of inhibitors detected in the Enceladian plume. These findings are important for understanding the eco-physiology of methanogens on Earth and have implications for the use of biomarkers as possible signs of extraterrestrial life for future space missions in the Solar System. Along similar lines, Taubner et al, 2018, discusses biological methane production under putative Enceladus-like conditions.

Further unrelated to biotechnology, Kengen & Stams 1994 relates to the formation of L-alanine as a reduced end product in carbohydrate fermentation by the hyperthermophilic archaeon Pyrococcus furiosus. Sment & Konisky, 1989, alleges excretion of amino acids by 1, 2, 4-triazole-3-alanine-resistant mutants of Methanococcus voltae in contrast to wild-type. In the experiments described in this document, M. voltae was grown under a H₂—CO₂ atmosphere in defined medium containing 19 amino acids.

Also unrelated to biotechnology, Porat et al, 2004, discusses biosynthetic pathways for aromatic amino acids in Methanococcus maripaludis.

Whitman et al., 1986, describes the isolation and characterization of 22 mesophilic methanococci. Cultures are grown under H₂—CO₂, while media are prepared under N₂—CO₂. The document is entirely silent on amino acid production.

Also unrelated to biotechnology, Fardeau et al., 1987, relates to the energetics of the growth of Methanobacterium thermoautotrophicum and Methanococcus thermolithotrophicus on ammonium chloride and dinitrogen. The document is entirely silent on amino acid production.

Along similar lines, Whitman et al, 1982, concerns the nutrition and carbon metabolism of Methanococcus voltae. Various nitrogen sources are discussed. It is stated that ammonia is required for growth of M. voltae in defined medium. Mineral requirements are also discussed in the document.

Rittmann et al, 2021, generally concerns the use of archaea in biotechnology.

Several studies discuss the use of methanogenic archaea in renewable energy production by reducing CO₂ with H₂ to CH₄ (Pappenreiter et al. 2019, Rittmann et al. 2018, Mauerhofer et al. 2018, Abdel Azim et al. 2018, Abdel Azim et al. 2017, Rittmann 2015, Mauerhofer et al. 2021, Rittmann et al, 2012). Liu et al., 2021, concerns the effects of different amino acids and their configurations on methane yield and biotransformation of intermediate metabolites during anaerobic digestion. WO 2012/110256 A1 discloses a method of converting carbon dioxide and hydrogen to methane by methanogenic microorganisms. WO 2014/128300 A1 relates to a method and system for producing methane using methanogenic microorganisms and applying specific nitrogen concentrations in the liquid phase.

WO 2017/070726 A1 relates to a method for determining the culture status of microbe cultures, such as cultures with hydrogenotrophic methanogenic microorganisms.

Hoffarth et al, 2019, concerns the effect of N2 on biological methanation in a continuous stirred-tank reactor with Methanothermobacter marburgensis. It is stated that N2 behaves like an inert gas. It is further taught in the document that “N2 does not participate in the catabolic reaction”. The document is entirely silent on amino acid production.

US 2011/281333 A1 relates to methane production from single-cell organisms, such as methanogens. The growth of methanogens includes consuming carbon dioxide to produce methane. Methods for enhancing the growth are disclosed. Gaseous N2 is not contemplated as a nitrogen source.

US 2018/0179559 A1 concerns a biological and chemical process utilizing chemoautotrophic microorganisms for the chemosynthetic fixation of carbon dioxide and/or other inorganic carbon sources into organic compounds and the generation of additional useful products. The microorganisms may be selected from many different bacterial and archaeal species.

EP 2 192 170 A1 relates to an amino acid-producing microorganism and method of producing amino acids. Disclosed is a microorganism (preferably selected from gamma-proteobacteria, coryneform bacteria or bacteria belonging to the genera Alicyclobacillus, Bacillus or the yeast Saccharomyces) which has an ability to produce an L-amino acid selected from the group consisting of L-lysine, L-threonine, L-tryptophan, L-phenylalanine, L-valine, L-leucine, L-isoleucine and L-serine and has been modified so that activity of pyruvate synthase or pyruvate: NADP⁺ oxidoreductase is increased.

WO 2016/179545 A1 and US 2018/0163240 A1 disclose compositions and methods for the biological production of methionine.

US 2019/0194630 A1 and US 2017/0130211 A1 relate to compositions and methods for biological production of amino acids in hydrogenotrophic microorganisms. In particular, the hydrogenotrophic microorganism may be chosen from Methanococcus and Methanosarcina.

WO 2020/252335 A1 relates to processes and systems for producing products by fermentation. Disclosed is in particular a process comprising (a) feeding a gaseous mixture comprising Coₓ and H₂, where x is 1 or 2, a nitrogen source, and optionally a sulfur source to a bioreactor that contains hydrogenotrophic microorganisms under conditions such that the hydrogenotrophic microorganisms produce at least one fermentation product chosen from an amino acid, an alcohol, aldehyde or ketone, a carboxylic acid, or a hydroxyl or keto acid; (b) removing a gas stream from the bioreactor, the gas stream having at least one compound chosen from a sulfur containing compound, a nitrogen containing compound, H₂, COₓ, and a hydrocarbon compound, where x is 1 or 2; (c) removing a liquid stream from the bioreactor comprising fermentation broth, hydrogenotrophic microorganisms, and fermentation product; and (d) separating the hydrogenotrophic microorganisms from the liquid stream and recycling the hydrogenotrophic microorganisms back to the bioreactor. The hydrogenotrophic microorganisms may be chosen from methanogenic archaea.

Despite these efforts, there is still a need for further development of fermentative methods with methanogenic microorganisms.

It is thus an object of the present invention to provide improved methods for producing amino acids by fermentation in a bioreactor with methanogenic microorganisms. These methods should make more efficient use of (natural) resources, be more environmentally friendly, lead to the reduction of greenhouse gas emissions, have an increased yield and/or overcome one or more disadvantages of amino acid production methods known in the art.

### SUMMARY OF THE INVENTION

The present invention provides a method for producing amino acids by fermentation in a bioreactor. The bioreactor comprises methanogenic microorganisms in a fermentation broth. This method comprises at least the steps of feeding a gaseous carbon source comprising carbon dioxide and/or carbon monoxide, a nitrogen source and preferably a sulfur source to the bioreactor under conditions such that the methanogenic microorganisms produce the amino acids; and harvesting at least a portion of the amino acids from the supernatant of the fermentation broth.

Typically, the harvesting is followed by purification methods to separate the amino acids from other constituents of the fermentation broth.

In an aspect, the present invention provides the use of methanogenic microorganisms (e.g. any of the methanogenic archaea disclosed herein, such as for instance Methanothermobacter, Methanothermococcus, Methanocaldococcus and Methanococcus) for producing amino acids, wherein the amino acids are harvested from the supernatant of the fermentation broth.

In another aspect, the present invention provides a fermentation broth comprising methanogenic microorganisms (e.g. any of the methanogenic archaea disclosed herein, such as for instance Methanothermobacter, Methanothermococcus, Methanocaldococcus and Methanococcus) and amino acids in the supernatant (i.e. the culture supernatant).

In yet another aspect, the present invention provides a bioreactor comprising this fermentation broth.

Agriculture and the production of artificial nitrogen-containing fertilizers are an indirect source of greenhouse gas emissions through releasing N20 via nitrification of ammonia (NH₃). The Haber-Bosch processes is the main industrial procedure for synthetic N2 fixation and responsible for a release of 1.5 tons of CO₂ per ton of NH₃ produced. In the course of the present invention, the inventors examined whether a biological process could be used for carbon and/or N2 fixation and concomitant amino acid production. Surprisingly, it turned out that methanogenic microorganisms actively excrete (or secrete) many different amino acids into the culture supernatant. The secretion of these amino acids to the supernatant is especially remarkable, as it simplifies downstream steps (e.g. no cell lysis required for harvesting the product) while at the same time increasing productivity (as methanogenic microorganisms remain viable and can remain or be fed back into the bioreactor).

### DETAILED DESCRIPTION OF THE INVENTION

The detailed description given below relates to all of the above aspects of the invention unless explicitly excluded.

According to another preferred embodiment, the method is a continuous process, a fed-batch process, a batch process, a closed batch process, a repetitive batch process, a repetitive fed-batch process or a repetitive closed batch process, preferably a continuous process, a fed-batch process or a repetitive fed-batch process, especially a continuous process. It is particularly preferred that the continuous process (culture) is a chemostat process (culture), especially with controlled pH.

Especially continuous culture, the harvesting step may include removing a liquid stream from the bioreactor, the liquid stream comprising fermentation broth, methanogenic microorganisms, and produced amino acids (in the supernatant of the fermentation broth), separating the methanogenic microorganisms from the liquid stream (e.g. by filtration) and recycling the methanogenic microorganisms back to the bioreactor. The harvested amino acids (which may be present in a liquid fraction of the fermentation broth) are then preferably further purified, e.g. by methods known in the art such as chromatography.

In the course of the present invention, it turned out that many of the methanogenic microorganisms remained viable and/or intact (as lysis was much reduced). It is thus preferred that the supernatant (used in the harvesting step) has a protein content of less than 1000 μg/mL, preferably less than 500 μg/mL, more preferably less than 250 μg/mL, even more preferably less than 100 μg/mL, yet even more preferably less than 50 μg/ml or even less than 40 μg/mL, especially less than 30 μg/mL or even less than 20 μg/mL. The protein content may be measured by methods known in the art, e.g. Bradford protein assay.

Along similar lines, is highly preferred that at least 50%, preferably at least 60%, more preferably at least 708, even more preferably at least 80%, especially at least 90% or even at least 95% of the methanogenic microorganisms remain viable and/or intact prior to and during the harvesting. This applies especially to the methanogenic microorganisms present in a liquid stream removed from the bioreactor (e.g. in continuous culture). The methanogenic microorganisms may then be recycled back to the bioreactor.

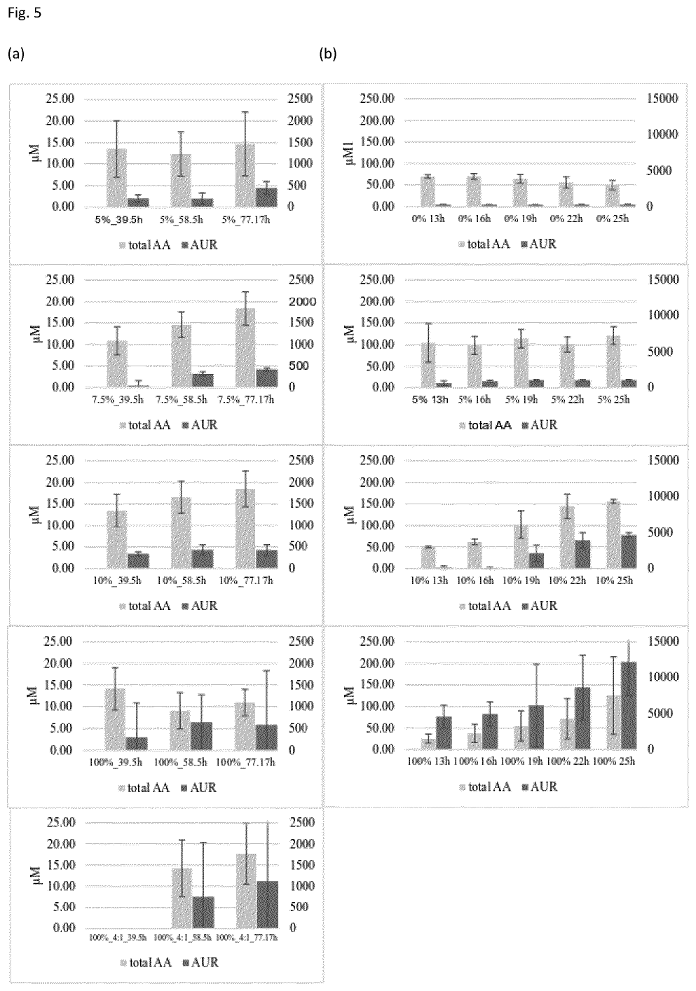

According to another preferred embodiment, the total amino acid concentration in the supernatant of the fermentation broth (used in the harvesting step) is at least 1 μmol/L, preferably at least 5 μmol/L, more preferably at least 10 μmol/L or even at least 25 μmol/L, even more preferably at least 50 μmol/L or even at least 100 μmol/L, especially at least 150 μmol/L (cf. Example 1 and FIG. 5). It is particularly preferred when the supernatant has a total amino acid concentration of at least 200 μmol/L, preferably at least 500 μmol/L, especially at least 1000 μmol/L (or even higher lower limits).

Biological molecular nitrogen (N2) fixation is a key process in the global nitrogen cycle where it is closely linked to the carbon cycle. With 16 moles ATP per mol N2 fixed, it is one of the most expensive metabolic processes (Thamdrup 2012; Hu and Ribbe 2012). Certain phyla of archaea, but also bacteria, are able to fix N2 (Fernandez et al. 2019). This biological process is referred to as diazotrophy. Diazotrophic growth was first reported among the archaea in 1984, when Methanosarcina barkeri (Murray and Zinder 1984) and Methanococcus thermolithotrophicus (Belay et al. 1984) were shown to be able to fix N2. The enzymes needed for the reduction of N2 are nitrogenases, which are encoded in the nif gene cluster (Raymond et al. 2004).

Jones & Stadtman, 1977, concerns the effects of selenium and tungsten on the growth of Methanococcus vannielii. The document teaches that growth of the microorganism on formate is markedly stimulated by selenium and tungsten. Along similar lines, Dridi et al., 2012, relates to tungsten-enhanced growth of Methanosphaera stadtmanae. Further, Lobo & Zinder, 1988, concerns diazotrophy and nitrogenase activity in the archaeon Methanosarcina barkeri 227. These documents are entirely silent on amino acid production.

When the nitrogen source comprises nitrogen gas, a certain ammonium concentration range has turned out to be especially advantageous for energy production (under N₂-fixing conditions). It is thus preferred that the ammonium concentration in the fermentation broth is 0.1 mmol/L to 200 mmol/L, preferably 2 mmol/L to 100 mmol/L, more preferably 4 mmol/L to 40 mmol/L, even more preferably 5 mmol/L to 35 mmol/L, yet even more preferably 6 mmol/L to 30 mmol/L, especially 7 mmol/L to 25 mmol/L, or even 10 mmol/L to 20 mmol/L. It is evident to the skilled person that, especially in continuous culture, the concentration in the broth may be subject to fluctuations (until a steady state is reached). It is however preferred that the ammonium concentration stays within any of the aforementioned ranges (e.g. 0.1 mmol/L to 200 mmol/L or 10 mmol/L to 20 mmol/L) for at least 5 min, preferably at least 10 min, even more preferably at least at least 20 min, yet even more preferably at least 1 h, especially at least 5 h or even at least 10 h (or at least 20 h or at least 40 h).

In addition, it is preferred when the tungstate concentration (in particular the orthotungstate, i.e. WO₄⁻², concentration) in the fermentation broth is below 0.1 μmol/L, preferably below 0.01 μmol/L, especially below 0.001 μmol/L (in particular, when the fermentation broth is essentially free of tungstate). In the course of the present invention, it turned out that this allows for more efficient amino acid production.

In a preferred embodiment, an electron donor (or electron donor compound) suitable for the methanogenic microorganisms is fed to the bioreactor. In particular, hydrogen gas (i.e. molecular hydrogen or H₂), acetate, a methyl compound preferably selected from methylamines, methyl sulfides and methanol, any other alcohol, preferably a secondary alcohol such as 2-propanol or 2-butanol, a methoxylated aromatic compound and/or formate are fed to the bioreactor (as electron donor compounds). Hydrogen gas, acetate, methanol or combinations thereof (e.g. methanol and acetate) are particularly preferred.

According to a further preferred embodiment, methane (produced by the methanogenic microorganisms) is harvested from the bioreactor.

The amino acids produced by the methods and uses disclosed herein may be either or both the D- or L-isomer. The amino acid may e.g. be 2-aminobutyrate, alanine, beta-alanine, arginine, aspartate, carnitine, citruline, cystine, dehydroalanine, glutamate, glutamine, glycine, hydroxyproline, isoleucine, leucine, lysine, methionine, norleucine, norvaline, ornithine, phenylalanine, proline, pyroglutamate, pyrroproline, pyrrolysine selenocysteine, selenomethionine, serine, homoserine, threonine, tryptophan, tyramine, tyrosine, or valine.

The (harvested) amino acids preferably comprise at least one, preferably at least two or even at least three, more preferably at least four or even at least five, even more preferably at least seven or even at least nine, yet more preferably at least 12 or even at least 15, yet even more preferably at least 17 or even at least 18, especially all of the 20 canonical amino acids (i.e. Asp, Glu, Asn, Ser, His, Gln, Gly, Thr, Arg, Ala, Tyr, Val, Met, Trp, Ile, Phe, Leu, Lys, Cys and Pro). It is especially preferred when the (harvested) amino acids comprise one or more of the following: essential amino acids (essential for human consumption), branched chain amino acids (BCAAs) and glutamate, preferably wherein it least 50 mol %, preferably at least 60 mol %, especially at least 70 mol % of the (produced or harvested) amino acids are essential amino acids, branched chain amino acids (BCAAs) or glutamate.

According to another preferred embodiment, the total amino acid production rate per volume (of the supernatant) of fermentation broth is at least 0.01 μmol L⁻¹ h⁻¹, preferably at least 0.05 μmol L⁻¹ h⁻¹, more preferably at least 0.1 μmol L⁻¹ h⁻¹, even more preferably at least 0.5 μmol L⁻¹ h⁻¹, yet even more preferably at least 1.0 μmol L⁻¹ h⁻¹, especially at least 5 μmol L⁻¹ h⁻¹ or even at least 10 μmol L⁻¹ h⁻¹ (or even higher).

According to yet another preferred embodiment, the total amino acid production rate per biomass is at least 0.1 μmol g⁻¹ h⁻¹, preferably at least 0.5 μmol g⁻¹ h⁻¹, more preferably at least 1.0 μmol g⁻¹ h⁻¹, even more preferably at least 5 μmol g⁻¹ h⁻¹, yet even more preferably at least 10 μmol g⁻¹ h⁻¹, especially at least 50 μmol g⁻¹ h⁻¹ or even at least 100 μmol g⁻¹ h⁻¹ (or even higher).

The methanogenic microorganisms used may be natural (e.g. natural isolates or laboratory strains obtained therefrom) or genetically manipulated. By way of example, genetic manipulation in methanogenic archeaea such as manipulation based on site-directed mutagenesis, selectable markers, transformation methods, and reporter genes, is available to the skilled person, see e.g., Sarmiento et al. 2011. CRISPR-based gene editing and other CRISPR-based genetic tools are also available to the skilled person, see. e.g. Nayak & Metcalf, 2017 and Dhamad & Lessner, 2020.

According to a particular preferred embodiment, the methanogenic microorganisms comprise archaea selected from any of Methanobacteriales, Methanococcales, Methanomicrobiales, Methanosarcinales, Methanopyrales, Methanocellales, Methanomassiliicoccales, and Methanonatronarchaeales, preferably Methanobacteriales and Methanococcales; more preferably selected from Methanobacteriaceae, Methanocaldococcaceae and Methanococcaceae; in particular selected from Methanothermobacter, Methanothermococcus and Methanococcus. Particularly preferred are Methanothermobacter marburgensis, Methanocaldococcus jannaschii, Methanococcus igneus and Methanocaldococcus villosus. Other suitable methanogenic archaea species are for instance: Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanocella arvoryzae, Methanocella conradii, Methanocella paludicola, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanomicrococcus blatticola, Methanoplanus endosymbiosus, Methanoplanus limicola, Methanoplanus petrolearius, Methanoregula boonei, Methanosaeta concilii, Methanosaeta harundinacea, Methanosaeta pelagica, Methanosaeta thermophila, Methanosarcina acetivorans, Methanosarcina barkeri, Methanosarcina mazei, Methanosarcina thermophila, Methanomicrobium mobile, Methanococcus aeolicus, Methanococcus maripaludis, Methanococcus vannielii, Methanococcus voltae, Methanothermococcus thermolithotrophicus, Methanopyrus kandleri, Methanothermobacter thermautotrophicum, Methanocaldococcus fervens, Methanocaldococcus indicus, Methanocaldococcus infernus, and Methanocaldococcus vulcanius.

Further archaeal species or strains suitable for the present invention are e.g. disclosed in following research articles: Leigh 2000; Fardeu et al. 1987; Belay et al 1984; Murray and Zinder 1984; Schönheit & Thauer, 1980; Blank et al. 1995; Bult et al 1996; Kessler et al, 1997, Mauerhofer et al, 2021; all of them included herein by reference. Strains may e.g. be obtained from the “Deutsche Sammlung für Mikroorganismen und Zellkulturen GmbH” (DSMZ) (Braunschweig, Germany).

The methanogenic microorganisms may be hydrogenotrophic, aceticlastic, methylotrophic (e.g. H₂-dependent methylotrophic) or methoxydotrophic.

A (defined) coculture of methanogenic microorganisms in the bioreactor has also turned out to be advantageous. Accordingly, the methanogenic microorganisms (in the bioreactor) preferably comprise at least two different species.

It is highly preferred that the fermentation is started with the methanogenic microorganisms in chemically defined fermentation medium.

In another preferred embodiment, the fermentation is performed under anaerobic conditions.

When the method is a continuous process (continuous culture), it is preferred that the dilution rate D is 0.001 h⁻¹ to 1.5 h⁻¹, preferably 0.01 h⁻¹ to 0.5 h⁻¹, especially 0.0125 h⁻¹ to 0.1 h⁻¹.

Nitrogen-fixing conditions and/or carbon-fixing conditions have turned out to be advantageous: Thus, according to a further preferment, at least 50%, preferably at least 60%, more preferably at least 70%, even more preferably at least 80%, yet even more preferably at least 90% or even more preferably at least 95%, especially at least 99% or even at least 99.9% of all nitrogen atoms of all nitrogen sources fed to the bioreactor are fed to the bioreactor in the form of nitrogen gas. In yet another preferred embodiment, at least 50%, preferably at least 60%, more preferably at least 70%, even more preferably at least 80%, yet even more preferably at least 90% or even more preferably at least 95%, especially at least 99% or even at least 99.9% of all carbon atoms of all carbon sources fed to the bioreactor are fed to the bioreactor in the form of carbon dioxide gas and/or carbon monoxide gas.

Typically, also a sulfur source is fed to be bioreactor. This source preferably comprises cysteine and/or sulfide, especially wherein the fermentation broth comprises sulfide at a concentration of 0.001-150 mg/L, preferably 0.01-100 mg/L, especially 1 to 80 mg/L and/or wherein the sulfide feed rate or cysteine feed rate is 0.0001-0.2 mol L⁻¹ h⁻¹, preferably 0.001-0.05 mol L⁻¹ h⁻¹.

A variety of different bioreactors may be used in the methods and uses disclosed herein. Liquid phase bioreactors (e.g. stirred tank, packed bed, one liquid phase, two liquid phase, hollow fiber membrane) are well known in the art. Multiphase bioreactors (e.g., bubble column bioreactor, trickle bed bioreactor (fixed or packed bed), fluidized bed bioreactor) may also be used. The bioreactor is typically partially made of (stainless) steel, plastic and/or glass. Usually, the bioreactor contains at least one inlet, which allows a gas or gas mixture to enter, and at least two outlets. One outlet allows for the removal of a liquid stream, which contains the one or more fermentation products (i.e. amino acids), and the other outlet allows for the removal of a gas stream (such as methane produced by the methanogenic microorganisms). In embodiments, the bioreactor is a chemostat.

There is also an increasing demand for non-proteinogenic amino acids. Norvaline (also called Nva herein) is a non-canonical amino acid which finds increasing use as a dietary supplement. Further, it is used for inducing vasodilation (see for instance U.S. Pat. No. 11,260,039 B1). It was also suggested as a therapeutic agent against Alzheimer's disease (Polis et al, 2019).

Not related thereto, norvaline has also gained interest when found incorporated into protein-based biopharmaceuticals produced by recombinant E. coli fermentation processes (Biermann et al, 2013) which is generally not desired.

CN 106520651 A discloses a method for producing L-norvaline through enzymatic transformation.

Kisumi et al, 1976, discusses the biosynthesis of the non-proteinogenic amino acids norvaline, norleucine and homoisoleucine in the enterobacterium Serratia marcescens.

US 2006/0057685 A1 relates to a fermentative method for producing norvaline using a bacterium of the Enterobacteriaceae family having all acetohydroxy acid synthases inactivated.

In the course of the present invention, it was surprisingly observed that methanogenic archaea are capable of norvaline production and active excretion of norvaline into the cell culture supernatant, in particular under carbon-fixing conditions (or carbon- and nitrogen-fixing conditions). Norvaline production has not been observed before in archaea, let alone in methanogenic archaea. This finding of the present invention enables combined carbon (and N₂) fixation and concomitant norvaline production.

Thus, in a particularly preferred embodiment, the (harvested) amino acids preferably comprise norvaline.

According to another preferred embodiment, the norvaline production rate per volume of fermentation broth is at least 0.001 μmol L⁻¹ h⁻¹, preferably at least 0.005 μmol L⁻¹ h⁻¹, more preferably at least 0.01 μmol L⁻¹ h⁻¹, even more preferably at least 0.05 μmol L⁻¹ h⁻¹, yet even more preferably at least 0.1 μmol L⁻¹ h⁻¹, especially at least 0.5 μmol L⁻¹ h⁻¹ or even at least 1.0 μmol L⁻¹ h⁻¹ (or even higher).

According to yet another preferred embodiment, the norvaline production rate per biomass is at least 0.01 μmol g⁻¹ h⁻¹, preferably at least 0.05 μmol g⁻¹ h⁻¹, more preferably at least 0.1 μmol g⁻¹ h⁻¹, even more preferably at least 0.5 μmol g⁻¹ h⁻¹, yet even more preferably at least 1.0 μmol g⁻¹ h⁻¹, especially at least 5 μmol g⁻¹ h⁻¹ or even at least 10 μmol g⁻¹ h⁻¹ (or even higher).

The present invention further relates to the following embodiments:

Embodiment 1. A method of producing amino acids by fermentation in a bioreactor, wherein the bioreactor comprises methanogenic microorganisms in a fermentation broth, the method comprising at least the steps of
feeding a gaseous carbon source comprising carbon dioxide and/or carbon monoxide, a nitrogen source and preferably a sulfur source to the bioreactor under conditions such that the methanogenic microorganisms produce the amino acids; and
harvesting at least a portion of the amino acids from the supernatant of the fermentation broth.

Embodiment 2. The method of embodiment 1, wherein the methanogenic microorganisms secrete the amino acids into the supernatant of the fermentation broth.

Embodiment 3. The method of embodiment 1 or 2, wherein the ammonium concentration in the fermentation broth stays within 0.1 mmol/L to 200 mmol/L, preferably 2 mmol/L to 100 mmol/L, more preferably 4 to 40 mmol/L, even more preferably 5 mmol/L to 35 mmol/L, yet even more preferably 6 mmol/L to 30 mmol/L, especially 7 mmol/L to 25 mmol/L, or even 10 mmol/L to 20 mmol/L for at least 5 min, preferably at least 10 min, even more preferably at least at least 20 min, yet even more preferably at least 1 h, especially at least 5 h or even at least 10 h (or at least 20 h or at least 40 h).

Embodiment 4. The method of any one of embodiments 1 to 3, wherein the fermentation broth comprises ammonium at a concentration of 0.1 mmol/L to 200 mmol/L, preferably 2 mmol/L to 100 mmol/L, more preferably 4 to 40 mmol/L, even more preferably 5 mmol/L to 35 mmol/L, yet even more preferably 6 mmol/L to 30 mmol/L, especially 7 mmol/L to 25 mmol/L, or even 10 mmol/L to 20 mmol/L.

Embodiment 5. The method of any one of embodiments 1 to 4, wherein the fermentation broth is essentially free of tungstate.

Embodiment 6. The method of any one of embodiments 1 to 5, wherein the fermentation broth is essentially free of orthotungstate.

Embodiment 7. The method of any one of embodiments 1 to 6, wherein an electron donor compound suitable for the methanogenic microorganisms is fed to the bioreactor.

Embodiment 8. The method of embodiment 7, wherein the electron donor compound is selected from hydrogen gas, acetate, a methyl compound (preferably selected from methylamines, methyl sulfides and methanol), any other alcohol (preferably a secondary alcohol such as 2-propanol or 2-butanol), a methoxylated aromatic compound, formate, and combinations thereof; preferably selected from hydrogen gas, acetate, methanol and combinations thereof.

Embodiment 9. The method of embodiment 7, wherein the electron donor compound comprises hydrogen gas.

Embodiment 10. The method of any one of embodiments 1 to 6, wherein hydrogen gas, acetate, a methyl compound (preferably selected from methylamines, methyl sulfides and methanol), any other alcohol (preferably a secondary alcohol such as 2-propanol or 2-butanol), a methoxylated aromatic compound and/or formate are fed to the bioreactor; preferably wherein hydrogen gas, acetate, methanol or combinations thereof are fed to the bioreactor.

Embodiment 11. The method of embodiment 10, wherein hydrogen gas is fed to the bioreactor.

Embodiment 12. The method of embodiment 10 or 11, wherein acetate and methanol are fed to the bioreactor.

Embodiment 13. The method of any one of embodiments 1 to 12, further comprising the step of harvesting at least a portion of the amino acids from the bioreactor.

Embodiment 14. The method of embodiment 13, wherein the harvesting comprises removing a liquid stream from the bioreactor, wherein the liquid stream comprises fermentation broth, methanogenic microorganisms, and produced amino acids (in particular in the supernatant), separating the methanogenic microorganisms from the liquid stream and recycling the methanogenic microorganisms back to the bioreactor; preferably wherein at least 50%, preferably at least 60%, more preferably at least 70%, even more preferably at least 80%, especially at least 90% or even at least 95% of the methanogenic microorganisms remain viable and/or intact.

Embodiment 15. The method of embodiment 14, further comprising the step of purifying the produced amino acids from the liquid stream, in particular from the supernatant of the fermentation broth; preferably wherein the total produced amino acid concentration in the supernatant of the fermentation broth is at least 1 μmol/L, preferably at least 5 μmol/L, more preferably at least 10 μmol/L or even at least 25 μmol/L, even more preferably at least 50 μmol/L or even at least 100 μmol/L, especially at least 150 μmol/L.

Embodiment 16. The method of any one of embodiments 1 to 15, wherein the nitrogen source comprises nitrogen gas.

Embodiment 17. The method of any one of embodiments 1 to 16, wherein the supernatant of the fermentation broth has a total protein content of less than 1000 μg/mL, preferably less than 500 μg/mL, more preferably less than 250 μg/mL, even more preferably less than 100 μg/mL, yet even more preferably less than 50 μg/ml or even less than 40 μg/mL, especially less than 30 μg/mL or even less than 20 μg/mL.

Embodiment 18. The method of any one of embodiments 1 to 17, wherein at least 50%, preferably at least 60%, more preferably at least 70%, even more preferably at least 80%, especially at least 90% or even at least 95% of the methanogenic microorganisms remain viable and/or intact prior to and/or during the harvesting.

Embodiment 19. The method of any one of embodiments 1 to 18, wherein the total amino acid concentration in the supernatant of the fermentation broth is at least 1 μmol/L, preferably at least 5 μmol/L, more preferably at least 10 μmol/L or even at least 25 μmol/L, even more preferably at least 50 μmol/L or even at least 100 μmol/L, especially at least 150 μmol/L.

Embodiment 20. The method of any one of embodiments 1 to 19, wherein the amino acids further comprise, or wherein said portion further comprises, at least one, preferably at least two or even at least three, more preferably at least four or even at least five, even more preferably at least seven or even at least nine, yet more preferably at least 12 or even at least 15, yet even more preferably at least 17 or even at least 18, especially all of the 20 canonical amino acids (in particular their L-form).

Embodiment 21. The method of any one of embodiments 1 to 19, wherein the amino acids comprises, or wherein said portion comprises, at least one, preferably at least two or even at least three, more preferably at least four or even at least five, even more preferably at least seven or even at least nine, yet more preferably at least 12 or even at least 15, yet even more preferably at least 17 or even at least 18, especially all of: 2-aminobutyrate, alanine, beta-alanine, arginine, aspartate, carnitine, citruline, cystine, dehydroalanine, glutamate, glutamine, glycine, hydroxyproline, isoleucine, leucine, lysine, methionine, norleucine, norvaline, ornithine, phenylalanine, proline, pyroglutamate, pyrroproline, pyrrolysine selenocysteine, selenomethionine, serine, threonine, tryptophan, tyramine, tyrosine, and valine (in particular their L-form).

Embodiment 22. The method of any one of embodiments 1 to 19, wherein the amino acids comprise, or wherein said portion comprises, ornithine and/or norleucine, in particular L-ornithine and/or L-norleucine.

Embodiment 23. The method of any one of embodiments 1 to 19, wherein the amino acids comprise, or wherein said portion comprises, one or more of the following: essential amino acids (essential for human consumption), branched chain amino acids (BCAAs) and glutamate (in particular their L-form); preferably wherein it least 50 mol %, preferably at least 60 mol %, especially at least 70 mol % of the (produced or harvested) amino acids are essential amino acids, branched chain amino acids (BCAAs) or glutamate (in particular their L-form).

Embodiment 24. The method of any one of embodiments 1 to 23, wherein the production rate of said amino acids per volume of fermentation broth is at least 0.01 μmol L⁻¹ h⁻¹, preferably at least 0.05 μmol L⁻¹ h⁻¹, more preferably at least 0.1 μmol L⁻¹ h⁻¹, even more preferably at least 0.5 μmol L⁻¹ h⁻¹, yet even more preferably at least 1.0 μmol L⁻¹ h⁻¹, especially at least 5 μmol L⁻¹ h⁻¹ or even at least 10 μmol L⁻¹ h⁻¹.

Embodiment 25. The method of any one of embodiments 1 to 24, wherein the total amino acid production rate per volume of fermentation broth is at least 0.01 μmol L⁻¹ h⁻¹, preferably at least 0.05 μmol L⁻¹ h⁻¹, more preferably at least 0.1 μmol L⁻¹ h⁻¹, even more preferably at least 0.5 μmol L⁻¹ h⁻¹, yet even more preferably at least 1.0 μmol L⁻¹ h⁻¹, especially at least 5 μmol L⁻¹ h⁻¹ or even at least 10 μmol L⁻¹ h⁻¹.

Embodiment 26. The method of any one of embodiments 1 to 25, wherein the production rate of said amino acids per biomass is at least 0.1 μmol g⁻¹ h⁻¹, preferably at least 0.5 mol g⁻¹ h⁻¹, more preferably at least 1.0 μmol g⁻¹ h⁻¹, even more preferably at least 5 μmol g⁻¹ h⁻¹, yet even more preferably at least 10 μmol g⁻¹ h⁻¹, especially at least 50 μmol g⁻¹ h⁻¹ or even at least 100 μmol g⁻¹ h⁻¹.

Embodiment 27. The method of any one of embodiments 1 to 26, wherein the total amino acid production rate per biomass is at least 0.1 μmol g⁻¹ h⁻¹, preferably at least 0.5 μmol g⁻¹ h⁻¹, more preferably at least 1.0 μmol g⁻¹ h⁻¹, even more preferably at least 5 μmol g⁻¹ h⁻¹, yet even more preferably at least 10 μmol g⁻¹ h⁻¹, especially at least 50 μmol g⁻¹ h⁻¹ or even at least 100 μmol g⁻¹ h⁻¹.

Embodiment 28. The method of any one of embodiments 1 to 27, wherein methane (produced by the methanogenic microorganisms) is harvested from the bioreactor.

Embodiment 29. The method of any one of embodiments 1 to 28, wherein the methanogenic microorganisms are genetically manipulated microorganisms.

Embodiment 30. The method of any one of embodiments 1 to 28, wherein the methanogenic microorganisms are natural isolates or laboratory strains obtained therefrom.

Embodiment 31. The method of any one of embodiments 1 to 30, wherein the methanogenic microorganisms comprise archaea.

Embodiment 32. The method of embodiment 31, wherein the archaea are selected from any of Methanobacteriales, Methanococcales, Methanomicrobiales, Methanosarcinales, Methanopyrales, Methanocellales, Methanomassiliicoccales, and Methanonatronarchaeales, preferably Methanobacteriales and Methanococcales; more preferably selected from Methanobacteriaceae, Methanocaldococcaceae and Methanococcaceae; in particular selected from Methanothermobacter, Methanothermococcus, Methanocaldococcus and Methanococcus.

Embodiment 33. The method of embodiment 31, wherein the archaea are selected from Methanothermobacter marburgensis, Methanocaldococcus jannaschii, Methanococcus igneus and Methanocaldococcus villosus.

Embodiment 34. The method of embodiment 31, wherein the archaea are of the genus Methanothermobacter, in particular of the species Methanothermobacter marburgensis.

Embodiment 35. The method of embodiment 31, wherein the archaea are selected from Methanothermobacter marburgensis, Methanocaldococcus jannaschii, Methanococcus igneus, Methanocaldococcus villosus, Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanocella arvoryzae, Methanocella conradii, Methanocella paludicola, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanomicrococcus blatticola, Methanoplanus endosymbiosus, Methanoplanus limicola, Methanoplanus petrolearius, Methanoregula boonei, Methanosaeta concilii, Methanosaeta harundinacea, Methanosaeta pelagica, Methanosaeta thermophila, Methanosarcina acetivorans, Methanosarcina barkeri, Methanosarcina mazei, Methanosarcina thermophila, Methanomicrobium mobile, Methanococcus aeolicus, Methanococcus maripaludis, Methanococcus vannielii, Methanococcus voltae, Methanothermococcus thermolithotrophicus, Methanopyrus kandleri, Methanothermobacter thermautotrophicum, Methanocaldococcus fervens, Methanocaldococcus indicus, Methanocaldococcus infernus, Methanocaldococcus vulcanius and combinations thereof.

Embodiment 36. The method of any one of embodiments 1 to 35, wherein the methanogenic microorganisms comprise at least two different species; preferably wherein at least one, especially at least two, of the species is archaeal, in particular selected from any of Methanobacteriales, Methanococcales, Methanomicrobiales, Methanosarcinales, Methanopyrales, Methanocellales, Methanomassiliicoccales, and Methanonatronarchaeales, preferably Methanobacteriales and Methanococcales, more preferably selected from Methanobacteriaceae, Methanocaldococcaceae and Methanococcaceae, in particular selected from Methanothermobacter, Methanothermococcus, Methanocaldococcus and Methanococcus.

Embodiment 37. The method of any one of embodiments 1 to 36, wherein the methanogenic microorganisms comprise hydrogenotrophic, aceticlastic, methylotrophic or methoxydotrophic microorganisms or combinations thereof.

Embodiment 38. The method of any one of embodiments 1 to 37, wherein the fermentation is started with methanogenic microorganisms in chemically defined fermentation medium.

Embodiment 39. The method of any one of embodiments 1 to 38, wherein the fermentation is performed under anaerobic conditions.

Embodiment 40. The method of any one of embodiments 1 to 39, wherein the method is a continuous process, a fed-batch process, a batch process, a closed batch process, a repetitive batch process, a repetitive fed-batch process or a repetitive closed batch process, preferably a continuous process, a fed-batch process or a repetitive fed-batch process, especially a continuous process.

Embodiment 41. The method of embodiment 40, wherein the method is a chemostat process.

Embodiment 42. The method of embodiment 40 or 41, wherein the dilution rate D is 0.001 h⁻¹ to 1.5 h⁻¹, preferably 0.01 h⁻¹ to 0.5 h⁻¹, especially 0.0125 h⁻¹ to 0.1 h⁻¹.

Embodiment 43. The method of any one of embodiments 1 to 42, wherein at least 50%, preferably at least 60%, more preferably at least 70%, even more preferably at least 80%, yet even more preferably at least 90% or even more preferably at least 95%, especially at least 99% or even at least 99.9% of all nitrogen atoms of all nitrogen sources fed to the bioreactor are fed to the bioreactor in the form of nitrogen gas.

Embodiment 44. The method of any one of embodiments 1 to 43, wherein at least 50%, preferably at least 60%, more preferably at least 70%, even more preferably at least 80%, yet even more preferably at least 90% or even more preferably at least 95%, especially at least 99% or even at least 99.9% of all carbon atoms of all carbon sources fed to the bioreactor are fed to the bioreactor in the form of carbon dioxide gas and/or carbon monoxide gas.

Embodiment 45. The method of any one of embodiments 1 to 44, wherein the sulfur source is fed to the bioreactor, preferably wherein the sulfur source comprises cysteine and/or sulfide, especially wherein the fermentation broth comprises sulfide at a concentration of 0.001-150 mg/L, preferably 0.01-100 mg/L, especially 1 to 80 mg/L and/or wherein the sulfide feed rate or cysteine feed rate is 0.0001-0.2 mol L⁻¹ h⁻¹, preferably 0.001-0.05 mol L⁻¹ h⁻¹.

Embodiment 46. The method of any one of embodiments 1 to 45, wherein the bioreactor is a liquid phase or multiphase bioreactor.

Embodiment 47. The method of any one of embodiments 1 to 46, wherein the bioreactor is any one of stirred tank, packed bed, one liquid phase, two liquid phase, hollow fiber membrane, bubble column, trickle bed or fluidized bed bioreactor.

Embodiment 48. The method of any one of embodiments 1 to 47, wherein the bioreactor is a chemostat.

Embodiment 49. The method of any one of embodiments 1 to 48, wherein the amino acids comprise, or wherein said portion comprises, norvaline, in particular L-norvaline; preferably wherein the fermentation broth comprises ammonium at a concentration of at least 0.1 mmol/L, preferably at least 2 mmol/L, more preferably at least 4 mmol/L, even more preferably at least 5 mmol/L, yet even more preferably at least 6 mmol/L, especially at least 7 mmol/L or even at least 10 mmol/L.

Embodiment 50. The method of any one of embodiments 1 to 49, wherein the norvaline (in particular L-norvaline) production rate per volume of fermentation broth is at least 0.001 μmol L⁻¹ h⁻¹, preferably at least 0.005 μmol L⁻¹ h⁻¹, more preferably at least 0.01 μmol L⁻¹ h⁻¹, even more preferably at least 0.05 μmol L⁻¹ h⁻¹, yet even more preferably at least 0.1 μmol L⁻¹ h⁻¹, especially at least 0.5 μmol L⁻¹ h⁻¹ or even at least 1.0 μmol L⁻¹ h⁻¹.

Embodiment 51. The method of any one of embodiments 1 to 50, wherein the norvaline (in particular L-norvaline) production rate per biomass is at least 0.01 μmol g⁻¹ h⁻¹, preferably at least 0.05 μmol g⁻¹ h⁻¹, more preferably at least 0.1 μmol g⁻¹ h⁻¹, even more preferably at least 0.5 μmol g⁻¹ h⁻¹, yet even more preferably at least 1.0 μmol g⁻¹ h⁻¹, especially at least 5 μmol g⁻¹ h⁻¹ or even at least 10 μmol g⁻¹ h⁻¹.

Embodiment 52. Use of methanogenic microorganisms for producing amino acids, wherein the amino acids are harvested from the supernatant of the fermentation broth, preferably wherein the methanogenic microorganisms secrete the amino acids into the supernatant of the fermentation broth.

Embodiment 53. The use of embodiment 52, wherein the fermentation broth is as defined in any one of embodiments 3 to 6, 17, 19 and 45.

Embodiment 54. The use of embodiment 52 or 53, wherein the methanogenic microorganisms are fed an electron donor compound, preferably as defined in any one of embodiments 7 to 9, a carbon source, a nitrogen source and preferably a sulfur source.

Embodiment 55. The use of any one of embodiments 52 to 54, wherein the amino acids are as defined in any one of embodiments 20 to 23 and 49.

Embodiment 56. The use of any one of embodiments 52 to 55, wherein the production rate is as defined in any one of embodiments 24 to 27, 50 and 51.

Embodiment 57. The use of any one of embodiments 52 to 56, wherein the methanogenic microorganisms are as defined in any one of embodiments 29 to 37.

Embodiment 58. A fermentation broth comprising amino acids in the supernatant and methanogenic microorganisms.

Embodiment 59. The fermentation broth of embodiment 58, wherein the fermentation broth further is as defined in any one of embodiments 3 to 6, 17, 19 and 45.

Embodiment 60. The fermentation broth of embodiment 58 or 59, further comprising an electron donor compound, preferably as defined in any one of embodiments 7 to 9, a carbon source, a nitrogen source and preferably a sulfur source.

Embodiment 61. The fermentation broth of any one of embodiments 58 to 60, wherein the amino acids are as defined in any one of embodiments 20 to 23 and 49.

Embodiment 62. The fermentation broth of any one of embodiments 58 to 51, wherein the methanogenic microorganisms are as defined in any one of embodiments 29 to 37.

Embodiment 63. A bioreactor comprising the fermentation broth of any one of embodiments 58 to 62.

The present invention is further illustrated by the following figures and examples, without being restricted thereto.

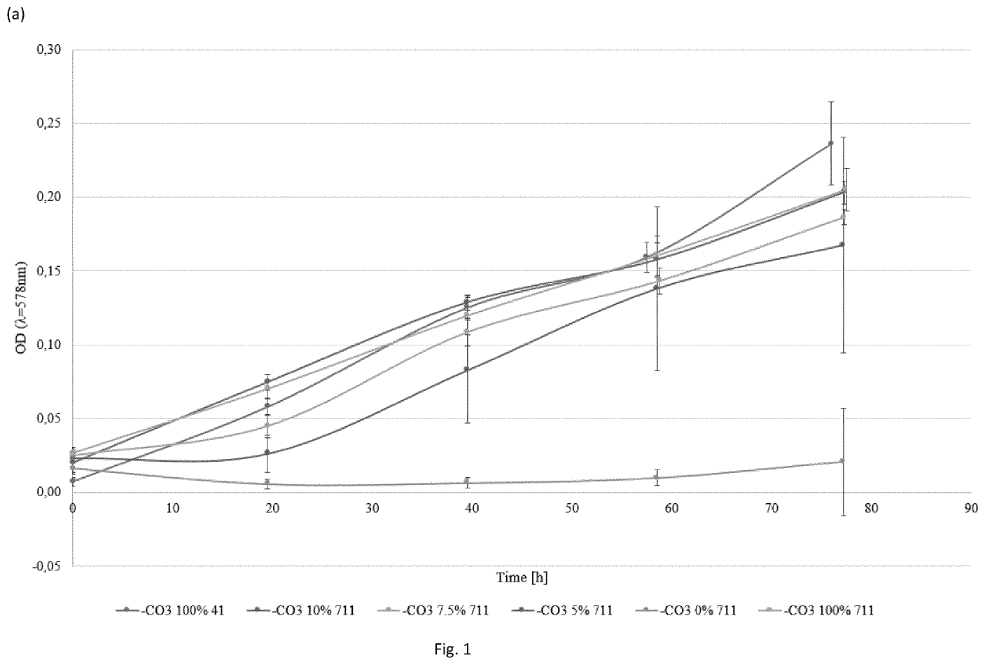

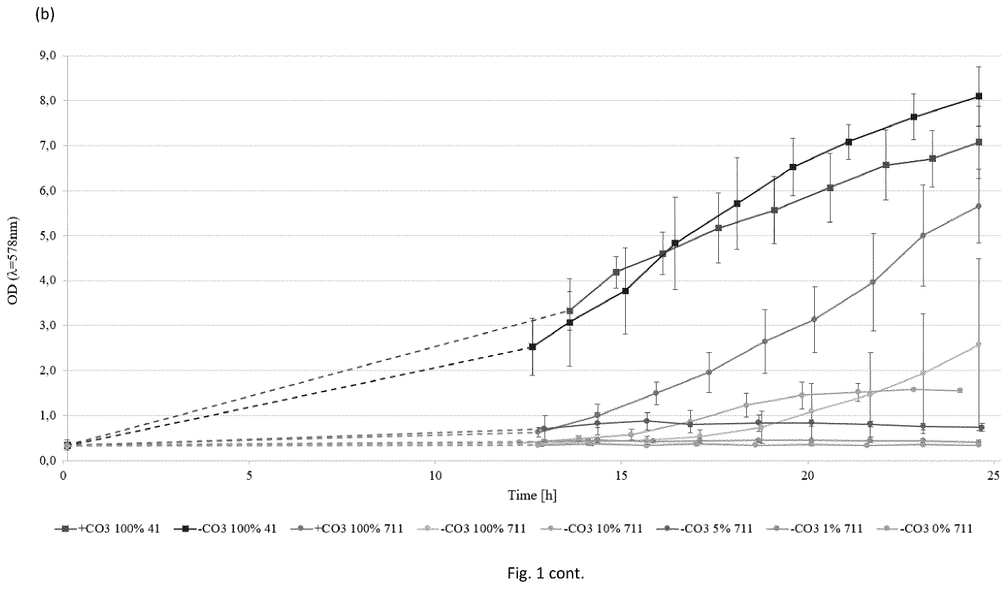

FIG. 1: Growth kinetics OD₅₇₈ₙₘ Of M. marburgensis with varying NH₄⁺ concentrations in relation to original medium gassed with H₂/CO₂/N₂ in the ratio 7:1:1 (711). Gassing in the ratio 4:1 (41) served as positive control. (a) Closed batch experiments with one washing step and gas samples taken after 40, 59 and 77 h. (b) Fed-batch experiments without washing. Dashed lines represent growth overnight. +/− indicates the presence of carbon in the media. 100% Reference runs were gassed with H₂/CO₂ in the ratio 4:1 (square). Gas samples were taken after approximately 0, 13, 16, 19, 22 and 25 h.

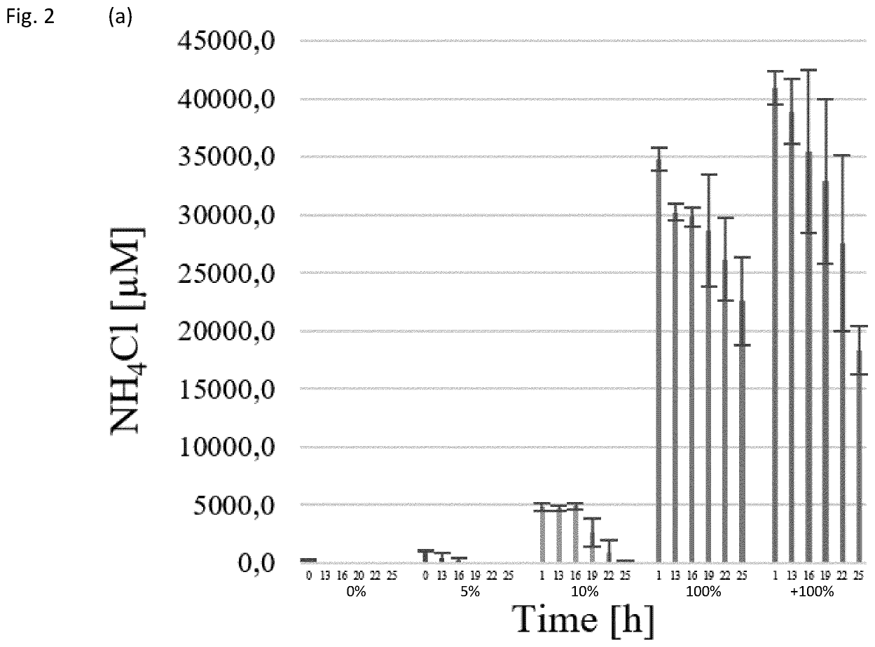

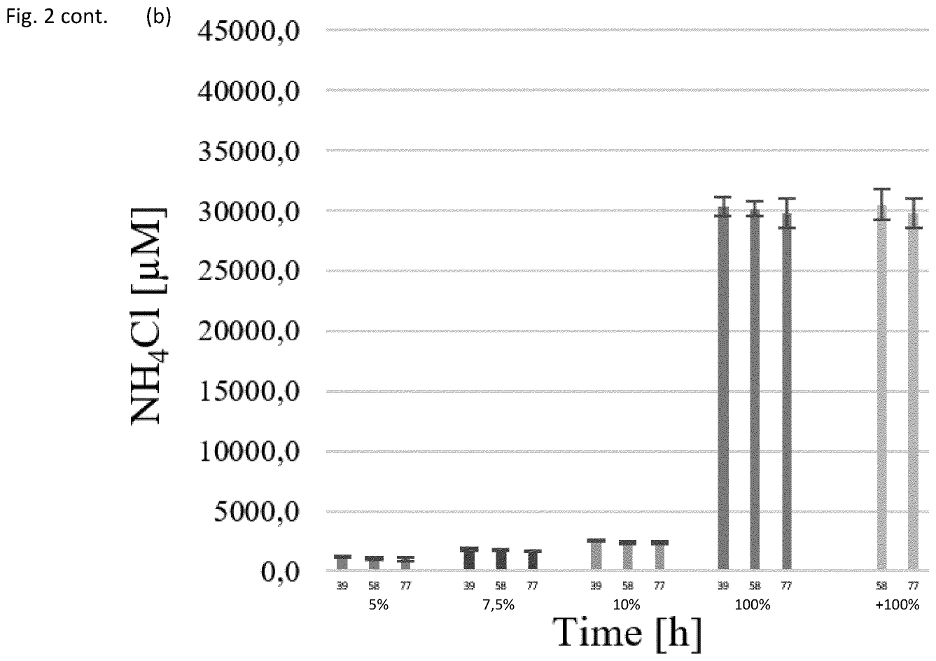

FIG. 2: NH₄⁺ concentrations in μM from (a) closed batch and (b) fed-batch experiments. Colours indicate the different concentrations of NH₄⁺in the media, 0% (orange), 5% (red), 7.5% (brown), 10% (yellow), 100% (grey), and 100% with carbon or 4:1 gassing (blue).

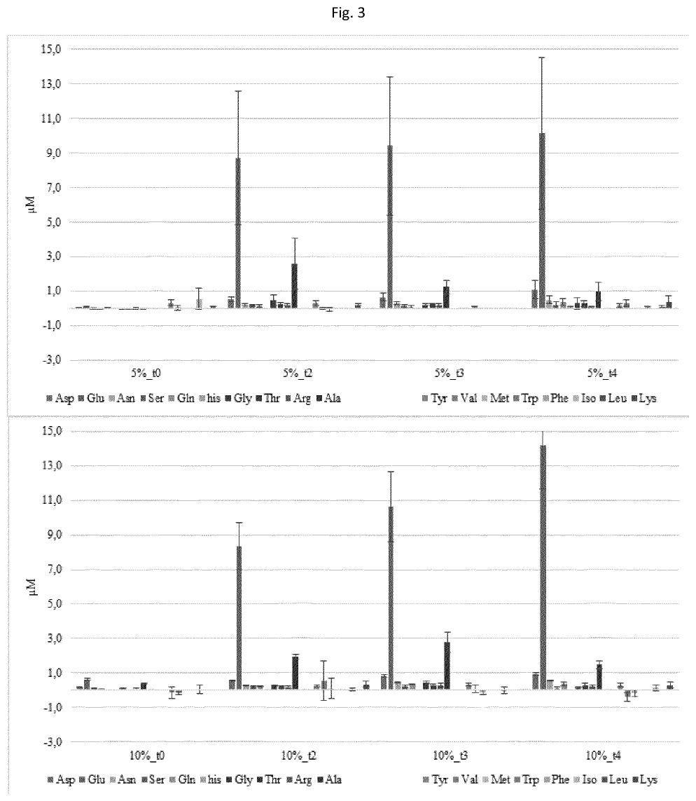

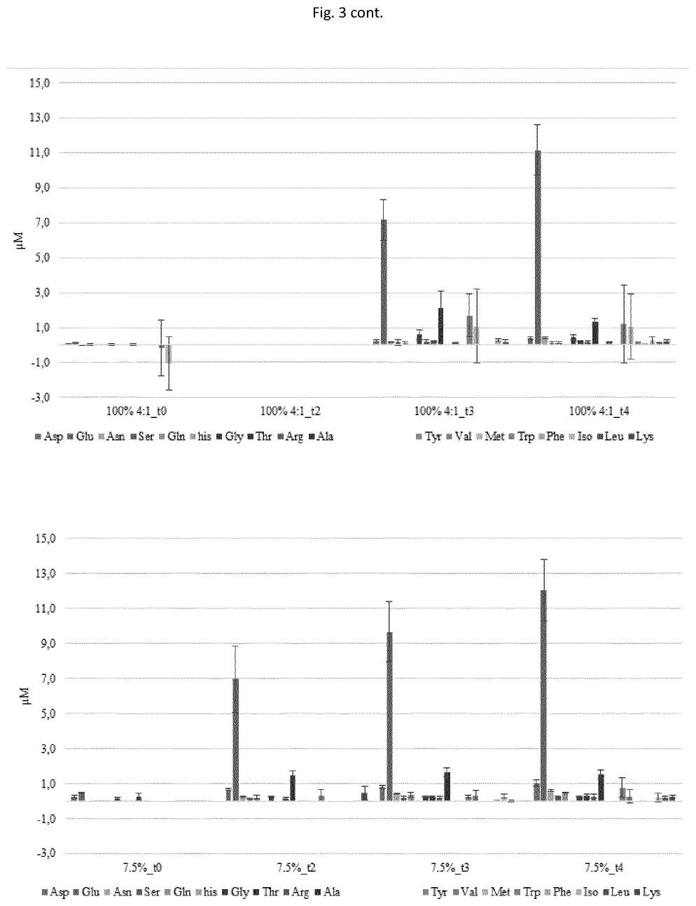

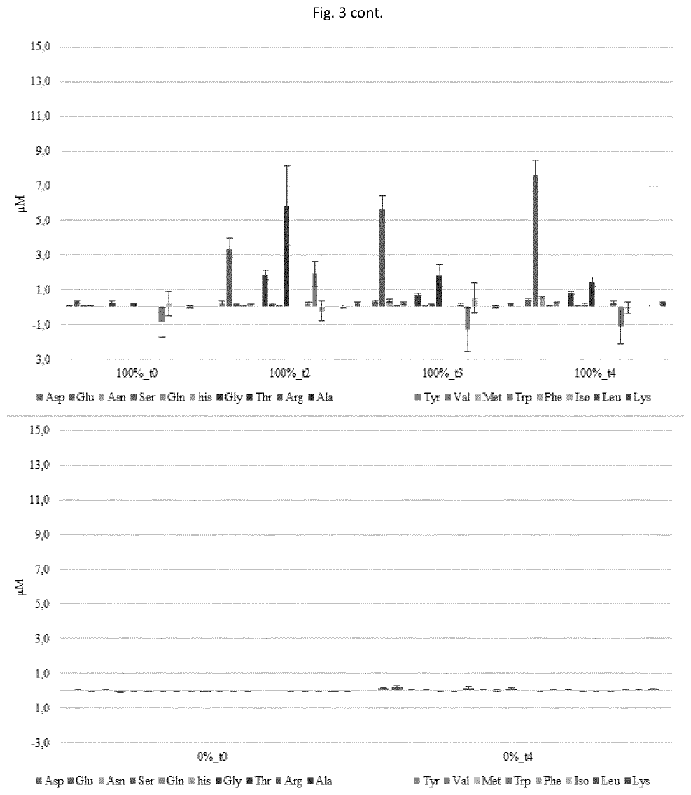

FIG. 3: Amino acid excretion of closed batch 5%, 7.5%, 10%, 100% and 0% experiments. Glutamic acid excretion was dominating.

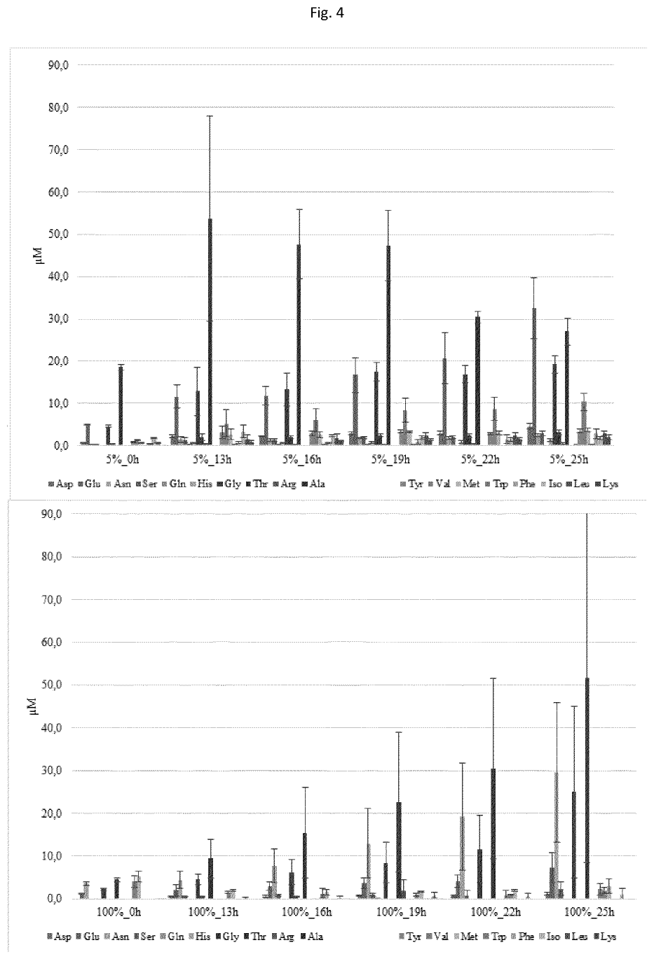

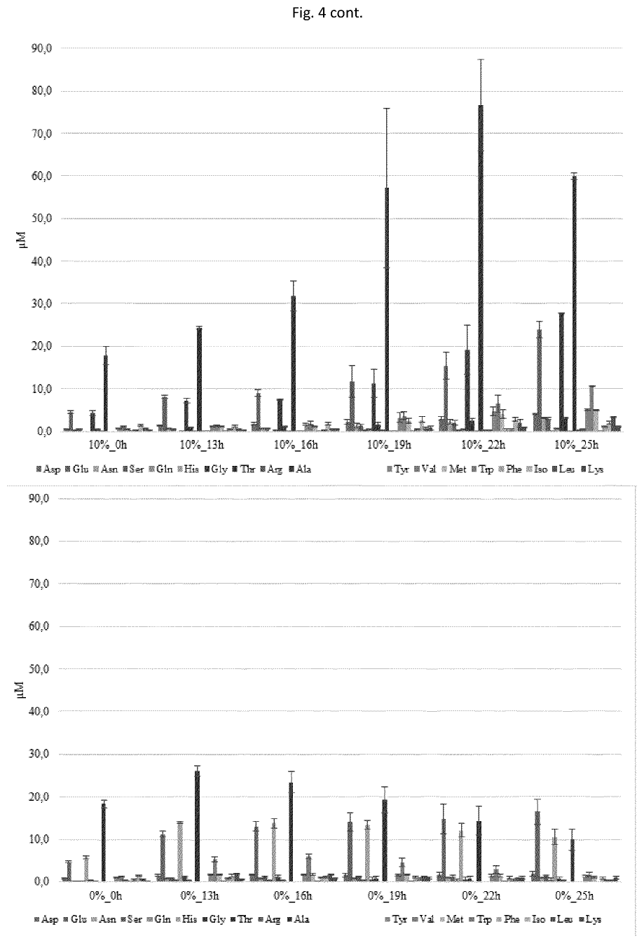

FIG. 4: Amino acid excretion of fed-batch 5%, 10%, 100% and 0% experiments. Alanine excretion is dominating.

FIG. 5: Total amount of excreted amino acids (total amino acids, AA) and ammonium uptake rate (AUR) of closed batch (a) and fed-batch (b) experiments. The amount of excreted AA is increasing with uptake of ammonium.

### EXAMPLES

### Example 1: Amino acid production and active secretion by Methanothermobacter marburgensis Under N₂-Fixing Conditions

The aim of our research was to examine the physiological and biotechnological characteristics of biological N₂ fixation in connection to H₂/CO₂ utilization of different methanogens.

Among several methanogens analyzed, Methanothermobacter marburgenis was prioritized to investigate N₂ fixation, CH₄ production, and amino acid excretion characteristics in closed batch and fed-batch cultivation modes and at different NH₄⁺ concentrations.

In brief, M. marburgensis was grown on chemically defined minimal medium with different concentrations of ammonium chloride (NH₄Cl) in a N₂/H₂/CO₂ atmosphere. This enabled the quantification of ammonia uptake, N₂ fixation, amino acid excretion and the conversion of H₂/CO₂ to CH₄. N₂ fixation by M. marburgensis was be confirmed in all experiments with H₂/N₂/CO₂ in the gas phase. Furthermore, the active excretion of proteinogenic amino acids was found, with highest detected values of glutamic acid, alanine, glycine and asparagin. The highest general production of 7.5 μmol L⁻¹ h⁻¹ was detected under “100%” NH₄⁺ concentration (see Table 1 below) in closed batch after 40 h. Hence, the concomitant production of amino acids and CH₄ from CO₂ turned out to be of biotechnological relevance in an integrated approach coupling biomethanation and N₂ fixation in a biorefinery concept.

### Materials and methods

### Strains

The following strains were selected for experiments: Methanothermobacter marburgensis (Schönheit et al. 1980; Wasserfallen et al. 2000), Methanobacterium thermaggregans (Blotevogel and Fischer 1985), Methanococcus maripaludis (Jones et al. 1983), Methanocaldococcus villosus (Bellack et al. 2011) and Methanothermococcus okinawensis (Takai et al. 2000). These strains may e.g. be obtained from the DSMZ, Braunschweig, Germany.

### Media

Pre-cultures of M. villosus and M. okinawensis, were grown on chemically defined medium according to Taubner & Rittmann, 2016. M. maripaludis was grown in McN medium (cf. Mauerhofer et al. 2021). Methanobacterium thermaggregans and Methanothermobacter marburgensis were cultivated on MM medium.

MM Medium (see also Table 1 below): NH₄Cl 2.1 g/L and KH₂PO₄ 6.8 g/L in ddH₂O. 200× trace element (TE) solution to be added (e.g. up to a concentration of 5 mL per L of MM medium): Titriplex I 9 g/L, add 800 mL H₂O and adjust the pH to 6.5 with 5 mol/L NaOH solution, then add (up to target concentration): MgCl₂·6H₂O 8 g/L, FeCl₂·4H₂O 2 g/L, CoCl₂·6H₂O 40 mg/L, NiCl₂·6H₂O 240 mg/L, NaMoO₄·2H₂O 40 mg, then adjust to pH 7.0 and volume of 1 L with 1 mol/L NaOH and ddH₂O. The medium may further contain e.g. 3.6 g/L NaHCO₃ as a carbon source. As a sulfur source, e.g. 2 mL of 0.5 mol/L Na₂S·9H₂O per liter may be added after anaerobization and autoclaving.

Surprisingly, it turned out that it was advantageous that the TE solution (and consequently, the medium) did not contain tungstate (or, at least, only below 0.1 μmol/L, preferably below 0.01 μmol/L, especially below 0.001 μmol/L), as amino acid yield under nitrogen fixing conditions was lower otherwise.

Medium was aliquoted into 117 mL serum bottles (VWR, Austria) to a total working volume of 50 mL, closed with blue rubber stoppers (pre-boiled for ten times 30 min, 20 mm, butyl rubber, Chemglass Life Sciences) and aluminum crimp caps (Ochs Laborbedarf, Bovenden, Germany). Sterile L-Cysteine-HCl. H₂O, sterile NaHCO₃ solution and Na₂S·9H₂O according to the media composition were added after autoclaving in an anaerobic glove box (Coy Laboratory Products, Grass Lake, USA). To ensure conditions are anaerobic the atmosphere in the headspace was changed by vacuuming and gassing with the respective gas (H₂/CO₂ or H₂/CO₂/N₂) mixture up to 2 bar rel. (3 bar abs.) repeating the procedure five times (Taubner & Rittmann 2016). For gassing, sterile syringe filters (w/0.2c μm cellulose, VWR International, USA) and sterile needles (disposal hypodermic needle, Gr 14, 0.60×30 mm, 23 G ×1¼″, Braun, Germany) were used.

Nitrogen-free (N-free) media were prepared by omitting NH₄Cl (2.1 g L⁻¹) and replace it with a chemically equal molar amount of NaCl (2.3 g L⁻¹) to ensure the correct salt concentration in the medium. To replace L-cysteine monohydrate, a diluted HCl solution was used to retrieve the pH value. MM medium with varying NH₄⁺ concentrations, was made as shown in Table 1. To ensure that CO₂ was the only carbon source, Na₂CO₃ was replaced by equal molarities of NaCl. Medium without Na₂CO₃ was manually adjusted to pH 6.8 by titrating 10 mol L⁻¹ NaOH.

TABLE 1
Composition of MM medium with varying NH₄⁺ concentration*

| | 100%+ | 100%− | 50%− | 25%− | 10%− | 5%− | 1%− | 0%− |
|---|---|---|---|---|---|---|---|---|
| g L⁻¹ | | | | | | | | |
| KH₂PO₄ | 6.8 | 6.8 | 6.8 | 6.8 | 6.8 | 6.8 | 6.8 | 6.8 |
| Na₂CO₃ | 3.6 | 0 | 0 | 0 | 0 | 0 | 0 | 0 |
| NH₄Cl | 2.1 | 2.1 | 1.05 | 0.525 | 0.21 | 0.105 | 0.021 | 0 |
| NaCl | 0 | 2 | 2.15 | 2.725 | 3.07 | 3.185 | 3.277 | 3.3 |
| mL L⁻¹ | | | | | | | | |
| TE 200x | 5 | 5 | 5 | 5 | 5 | 5 | 5 | 5 |

*in relation to original MM medium in g L⁻¹. Percentage of NH₄Cl in relation to original MM medium (which already contains 2.1 g L⁻¹ NH₄Cl).
−/+ presents the absence and presence of Na₂CO₃.
TE: trace element
solution

### Chemicals

H₂ (99.999%), CO₂ (99.999%), N₂ (99.999%), H₂/CO₂ (80%/20%), H₂/CO₂/N₂ (77.74%/11.13%/11.13%) were used for closed batch and fed-batch experiments. For gas chromatography (GC), N₂/CO₂ (80%/20%), CH₄ (99.995%) and the standard test gas (Messer GmbH, Wien, Austria) (containing 0.01 Vol.-% CH₄, 0.08 Vol.-% CO₂ in N₂) was additionally used. All gases, except the standard test gas, were purchased from Air Liquide (Air Liquide GmbH, Schwechat, Austria). All other chemicals were of highest grade available.

### Closed Batch Experiments

Cultures were incubated in a water bath (Burgwedel, Germany) at 65° C. (M. marburgensis, M. thermaggregans and M. okinawensis) or in a shaking air incubator at 37° C. (M. maripaludis) (Burgwedel, Germany) and 80° C. (M. villosus) (LABWIT Scientific Pty Ltd, Australia). For the purpose of N₂ fixation, all closed batch experiments were performed in a H₂/N₂/CO₂ atmosphere. For selecting the strain for prioritization, M. marburgensis, M. maripaludis, M. thermaggregans, M. villosus and M. okinawensis were grown in triplicates (n=3) with one zero control to an OD₅₇₈ of approximately 0.7. As M. marburgensis had one of the highest NH₄⁺ concentrations in the media, it was additionally examined if growth was affected by reducing the amount of NH₄⁺to one tenth of the original media concentration. To remove residual nitrogenous compounds from the media, pre-culture cells were washed before inoculation. For a complete N-free media all cultures were washed three times, for all other experiments one or no time. Experiments of only M. marburgensis were performed in quadruplicates (n=4) or octuplicates (n=8) at different NH₄⁺ concentrations (“0%”, “1%”, “10%”, “25%”, “50%” and “100%”, cf. Table 1 above) in relation to original media composition of 2.1 g L⁻¹. A pre-culture with one tenth of NH₄⁺served as inoculum. The batch with 0% served as negative control and 100% gassed with H₂/CO₂ in the ratio 4:1 as positive control.

After every incubation time the serum bottles were left at room temperature for 45 min to cool down. Pressure was measured with a digital manometer (Keller GmbH, Winterthur, Switzerland). Growth was measured spectrophotometrically via OD (λ=578 nm, blanked with Milli-Q water) (Beckman Coulter, California, USA). Liquid samples of 1 mL were taken and centrifuged at full speed (13200 rpm) for 30 min. Cell pellets and supernatant of each experiment were stored in sterile Eppendorf tubes until further analysis at −20° C.

### Fed-Batch Experiments

All fed-batch experiments were performed with M. marburgensis in triplicates with H₂/CO₂/N₂ gassing at a ratio of 7:1:1 in a DASGIP® 2.2 L bioreactor system (SR1500ODLS, Eppendorf AG, Hamburg, Germany) with 1.5 L working volume of MM medium including 100 μl L⁻¹ of antifoam (Struktol SB2023, Schill und Seilacher, Hamburg, Germany). Best growth conditions are pH of 7 and a temperature of 65° C. Gassing of N₂ and CO₂ was controlled via the MX4/4 unit (Eppendorf AG, Hamburg, Germany). H₂ gas flow was controlled via the C100 L Unit (Sierra Instruments, Monterey, USA). Gassing was performed with the same ratio as closed batch experiments. Redox potentials and pH values were monitored by individual redox- and pH-probes (Mettler Toledo GmbH, Wien, Austria). Every fed-batch cultivation was performed with the inoculum of a stock culture of M. marburgensis, adapted to fed-batch cultivation. Before inoculation, the bioreactor was gassed with H₂/N₂/CO₂ to ensure conditions are anaerobic and 5 mL of 0.5 mol L⁻¹ Na₂S. 9H₂O were added. Immediately after inoculation of 30 mL, feeding of 0.2 mL h⁻¹ 0.5M Na₂S·9H₂O was started and the agitation speed was set to 1600 rpm. Gaseous samples were taken after approximately 0, 13, 16, 19, 22 and 25 h. Batches with and without Na₂CO₃ in the media, performed under H₂/CO₂ atmosphere (ratio 4:1) served as reference. For analyzing growth and amino acid excretion, liquid samples were taken and treated as described above.

### Ammonium Determination

NH₄⁺ determination was performed using a modified procedure according to the method described before (Kandeler 1988). The oxidation solution, the colour reagent and the NH₄Cl stock solution were prepared freshly before the measurement. As standards, nine different concentrations, ranging from 100 μmol L⁻¹ to 1000 μmol L⁻¹ of NH₄Cl, were prepared. Samples were diluted with MilliQ to an end concentration between the standard ranges. Before the measurement, 300 μL of colour reagent and 120 μL oxidation solution were added immediately to the standards and samples and shortly mixed. After 30 min in the dark, measurement (>=660 nm) was performed using a 96 well plate (Microtest Plate 96 Well, F, Sarstedt A G & CO, Nümbrecht, Germany) with a plate photometer (Sunrise plate reader, Tecan Group AG, Mannedorf, Switzerland). Regression curve R² was always higher than 0.999.

### Gas Chromatography

Closed batch experiments and the off-gas composition (H₂, CO₂, CH₄ and N₂) of the collected gas samples from fed-batch experiments were analyzed by using the Agilent Gas Chromatograph (Agilent 7890A GC, Agilent Technologies, Santa Clara, CA, USA) equipped with a thermal conductivity detector (TDC) and a 19808 Shin Carbon ST Micropacked Column (Restek GmbH, Bad Homburg, Germany).

### Amino Acid Analysis

For amino acid analyses, supernatant of samples (obtained as described above) were diluted with Mill-Q water at the ratio of 1:4. Measurements were performed on Agilent 1260 Infinity Bioinert HPLC system containing a fluorescence detector, a column oven, an autosampler and a quaternary pump. 1 mL of sample was mixed with 75 μL borate buffer (0.4 N in water, pH=10.2; Agilent Technologies) followed by 5 μL OPA reagent, (3-mercaptopropionic acid in 0.4 mol L⁻¹ borate buffer and 10 mg mL⁻¹ of o-phthalaldehyde (OPA); Agilent Technologies). 100 μL of the mixture was injected to HPLC system after 2 min at 27° C. Fluorescent derivates (primary dissolved free amino acids) were separated at 25° C. and a flow rate of 0.8 mL min-1 on a Zorbax ECLIPSE AAA column (4.6×150 mm, 3.5 μm particle size, Agilent Technologies) with a Zorbax ECLIPSE AAA guard cartridge (4.6 ×150 mm, 5 μm particle size, Agilent Technologies). Excitation wavelength was 340 nm and emission 450 nm. The use of a gain factors 9 or 10 was depended on the expected concentration and pre-tested before. For identification and quantification of peaks a primary amino acid standard mix (AAS18, Sigma Aldrich) in different concentrations was prepared for each run according to the concentration range of the samples (100 nmol L⁻¹ to 15 μmol L⁻¹). AAS18 standard mix lacks five amino acids (asparagine (Asn), glutamic acid (Glu), gamma-aminobutyric acid (GABA), taurine (Tau), tryptophane (Trp); Sigma Aldrich) which were added. In total 20 different AA could be measured with this method. Valine and Methionine were excluded from evaluation as they are located within a signal noise “ammonium peak” and therefore hard to measure within experiments with high NH₄⁺ concentrations. The details of this method were as published before (Taubner et al. 2019).

### GC Analysis

The relative pressure in bar within the serum bottle was measured with a digital manometer (Keller GmbH, Winterthur, Switzerland). Gaseous substance (n/mol) in the serum bottles headspace was calculated via the ideal gas law. Headspace volume was determined in earlier experiments and adjusted after every OD measurement by the extracted sample volume of 0.75 mL. All measurements were performed at room temperature (25° C.).

To obtain the actual amount of N₂ the pressure inside the serum bottles was multiplied by 0.11392 based on the exact percentage of N₂ (11.392 Vol.-%) in the gas mixture, then multiplied with the normalized gas composition gained from GC measurement. The values of the zero-control served as N₂ baseline. Molecular N₂ uptake rate (NUR/mmol L⁻¹ h⁻¹) was calculated by dividing the deviation of N₂ before and after incubation (ΔN₂) by volume of the liquid medium and the time since last incubation (Δt):

$$NUR [ \frac{mmol}{L \cdot h} ] = \frac{{N}_{2} [ mol ]}{V o l . ( med ) [ L ] \cdot t [ h ]}$$

The quantitative/specific nitrogen uptake (qN₂/mmol h⁻¹ g 1) was determined by dividing the NUR by the biomass concentration (x/g L⁻¹) calculated with an experimentally determined coefficient:

$$q {N}_{2} [ \frac{mmol}{h \cdot g} ] = \frac{NUR}{x}$$

Carbon dioxide uptake rate (CUR/mmol L⁻¹ h⁻¹), molecular hydrogen uptake rate (HUR/mmol L⁻¹ h⁻¹), CH₄ evolution rate (MER/mmol L⁻¹ h⁻¹), carbon balance (C-balance), yields (Y_{(CH4/CO2)} and Y_{(x/CO2)}) and biomass productivity (rₓ/c-mmol L⁻¹ h⁻¹) was calculated as described elsewhere (Taubner et al. 2016; Rittmann et al. 2012, Bernacchi et al. 2014). The concentrations of H₂, CO₂, N₂ and CH₄ after GC measurements were obtained.

### Results

### Prioritization of Strains

Growth of M. marburgensis, M. maripaludis S0001, M. thermaggregans, M. villosus and M. okinawensis was analyzed in a H₂/CO₂/N₂ atmosphere in defined, but NH₄⁺ containing medium, and a N-free medium. This allowed us to screen for NH₄⁺ uptake, N₂ fixation, amino acid excretion and the conversion of H₂/CO₂ to CH₄ in parallel. All methanogens but M. thermaggregans could be grown to an OD₅₇₈ of 0.7 in NH₄⁺ containing medium. Further experiments showed that a certain amount of NH₄⁺was necessary for growth under these conditions. Due to most favorable growth characteristics in these experiments, M. marburgensis was selected for further experiments. Growth at specific NH₄⁺ concentrations with and without bicarbonate (Na₂CO₃) was already performed and showed no or nitrogen-limited growth in 0% and 1% and similar growth in all other concentrations. In experiments with Na₂CO₃ in the media, a higher growth rate was shown. NH₄⁺ uptake kinetics of M. marburgensis

Closed batch experiments of M. marburgensis, were then performed with 0%, 5%, 7.5%, 10% and 100% NH₄⁺in relation to original media composition of 2.1 g L⁻¹, gassed with H₂/CO₂/N₂ in the ratio 7:1:1. To reduce the possibility of a NH₄⁺carryover the experiments were performed with one washing step in octuplicates (n=8) with additional zero controls. Due to the biomass washing step a slower growth compared to non-washed biomass experiments was observed. After 77.17 h the OD₅₇₈ located between 0.17 and 0.20. The 100% 4:1 positive control as expected showed the best OD₅₇₈ value around 0.25. Gas samples were taken after approximately 40, 59 and 77 h (FIG. 1).

Additional fed-batch experiments (n=3) at NH₄⁺ concentrations of 0%, 1%, 5%, 10% and 100% were performed. Reference runs showed similar growth up to an OD₅₇₈ of 7.0 and 8.1 (FIG. 1). Comparing the runs at 100% NH₄⁺, a 2.2-fold higher OD₅₇₈ was obtained with Na₂CO₃ in the media. This effect was also visible in closed batch experiments. NH₄⁺ concentration of 10% showed a stagnation after 20 h reaching a final OD₅₇₈ Of 1.6, 5% displays stagnation after 15 h reaching an OD₅₇₈ of 0.9.

A detailed description of H₂ and CO₂ uptake rates, HUR and CUR, respectively, and MER is shown in Table 2.

TABLE 2
Hydrogen Uptake Rate (HUR), Carbon dioxide Uptake Rate (CUR), Nitrogen Uptake Rate (NUR) and Methane Evolution
Rate (MER) and C-Balance of closed batch experiments of M. marburgensis with different NH₄⁺ concentrations.

| | HUR | CUR | NUR | MER | Δx | | | Y | Y | C |
|---|---|---|---|---|---|---|---|---|---|---|
| | [mmol/L* h] | [mmol/L* h] | [mmol/L* h] | [mmol/L* h] | [g/L] | Δt | rx | (CH₄/CO₂) | (x/CO₂) | Balance |
| 5%_39.5 h | −3.19 | −0.55 | 0.03 | 1.25 | 0.02 | 20.00 | 0.00 | 2.27 | 0.00 | 2.27 |
| 5%_58.5 h | −3.48 | −1.02 | 0.21 | 1.50 | 0.04 | 19.00 | 0.00 | 1.48 | 0.00 | 1.48 |
| 5%_77.17 h | −2.66 | −0.96 | −0.46 | 1.45 | 0.06 | 18.67 | 0.00 | 1.51 | 0.00 | 1.51 |
| 7.5%_39.5 h | −1.53 | −0.54 | −0.91 | 1.14 | 0.04 | 20.00 | 0.00 | 2.12 | 0.00 | 2.12 |
| 7.5%_58.5 h | 1.41 | −0.26 | 0.24 | 1.08 | 0.05 | 19.00 | 0.00 | 4.10 | 0.00 | 4.10 |
| 7.5%_77.17 h | −2.58 | −0.59 | −0.47 | 1.21 | 0.07 | 19.00 | 0.00 | 2.07 | 0.00 | 2.07 |
| 10%_39.5 h | −2.63 | −0.88 | −0.30 | 0.94 | 0.04 | 20.00 | 0.00 | 1.06 | 0.00 | 1.06 |
| 10%_58.5 h | −3.37 | −0.79 | −0.88 | 1.32 | 0.05 | 19.00 | 0.00 | 1.68 | 0.00 | 1.68 |
| 10%_77.17 h | −4.10 | −0.60 | −0.49 | 1.26 | 0.06 | 18.75 | 0.00 | 2.11 | 0.00 | 2.11 |
| 100%_39.5 h | −3.29 | −0.45 | −0.83 | 0.91 | 0.03 | 20.00 | 0.00 | 2.03 | 0.00 | 2.03 |
| 100%_58.5 h | −3.73 | −0.73 | −0.54 | 0.97 | 0.05 | 19.25 | 0.00 | 1.33 | 0.00 | 1.33 |
| 100%_77.17 h | −3.32 | −0.84 | −0.55 | 1.13 | 0.06 | 18.50 | 0.00 | 1.34 | 0.00 | 1.34 |

The interplay of simultaneous uptake of NH₄⁺ and N₂

An NH₄⁺ uptake during N₂ fixation is evident in closed batch and fed-batch experiments. Comparing FIG. 2 (chart a) to FIG. 2 (chart b) it is visible that during in fed-batch cultivation an NH₄⁺ limitation was observed at 0-10% of NH₄⁺, whereas in closed batch cultivation NH₄⁺was never completely consumed. Further, it is noticeable that within the closed batch experiments the NH₄⁺ concentration does not differ much between time points, in contrast to fed-batch experiments where NH₄⁺ decreases over time (FIG. 2). The highest consumption was achieved in the positive control experiments. In case of closed batch experiments the highest ammonia uptake rate (AUR) was achieved within the 100% 4:1 run with 243.8 μmol L⁻¹ h⁻¹ at a qN₂ of 4.6 μmol h⁻¹ g⁻¹, and in case of fed-batch experiments the highest AUR of 577.3 μmol L⁻¹ h⁻¹ and qN₂ of 10.8 μmol h⁻¹ g⁻¹ in 100% was obtained with bicarbonate in the media.

Highest NUR was calculated from 7.5% and 100% closed batch experiments after 40 h with 0.91 or 0.83 mmol L⁻¹ h⁻¹ respectively and from 10% after 59 h with 0.88 mmol L⁻¹ h⁻¹. The qN₂ shows the same pattern (Table 3). Earlier sampling time points showed higher NUR, later time points lower, but more balanced NUR values.

TABLE 3
NUR [mmol L⁻¹ h⁻¹] and qN₂ [mmol h⁻¹ g⁻¹] of
closed batch experiments in 5%, 7.5%, 10% and 100% NH₄Cl.

| | NUR | NUR SD | qN₂ | qN₂ SD | AUR | AUR SD |
|---|---|---|---|---|---|---|
| | [mmol L⁻¹ | [mmol L⁻¹ | [mmol h⁻¹ | [mmol h⁻¹ | [μmol L⁻¹ | [μmol L⁻¹ |
| | h⁻¹] | h⁻¹] | g⁻¹] | g⁻¹] | h⁻¹] | h⁻¹] |
| 0%_77 h | 0.83 | 0.10 | 298.31 | 164.27 | −1 | 0 |
| 5%_40 h | 0.25 | 0.09 | 12.50 | 7.52 | 73 | 31 |
| 5%_59 h | 0.23 | 0.13 | 5.52 | 3.96 | 54 | 48 |
| 5%_77 h | 0.46 | 0.24 | 7.47 | 3.81 | 97 | 52 |
| 7.5%_40 h | 0.91 | 0.44 | 27.08 | 13.73 | 13 | 41 |
| 7.5%_59 h | 0.27 | 0.11 | 5.53 | 2.57 | 87 | 17 |
| 7.5%_77 h | 0.47 | 0.08 | 7.19 | 1.40 | 92 | 12 |
| 10%_40 h | 0.30 | 0.10 | 10.74 | 3.25 | 122 | 16 |
| 10%_59 h | 0.88 | 0.12 | 17.05 | 2.47 | 117 | 42 |
| 10%_77 h | 0.49 | 0.19 | 7.65 | 2.95 | 92 | 44 |
| 100%_40 h | 0.83 | 0.13 | 29.94 | 5.91 | 109 | 285 |
| 100%_59 h | 0.54 | 0.14 | 11.84 | 2.97 | 177 | 229 |
| 100%_77 h | 0.55 | 0.19 | 9.30 | 3.30 | 128 | 453 |

Amino acid excretion by M. marburgensis

Active amino acid excretion by M. marburgenis was investigated in closed batch (FIG. 3) and fed-batch (FIG. 4) experiments. Independent of the cultivation mode almost all detectable amino acids were found. The highest excreted amino acids were glutamic acid (Glu), alanine (Ala), glycine (Gly) and asparagin (Asn). The concentrations of Glu, Gly and Asn were constantly increasing during the course of the cultivation, yet Ala was consumed after a certain timepoint. All cultivation experiments showed a clear NH₄⁺ dependendence where amino acid excretion in 5%, 7.5% and 10% varies from 100%. (FIGS. 3 and 4). Taking a closer look to volumetric values, in closed batch the highest value was obtained with glutamic acid with up to 4.59 μmol L⁻¹ h⁻¹ in 5% after 40 h and a smaller amount of alanine with up to 1.36 μmol L⁻¹ h⁻¹. Highest value of Gly was achieved in 100% after 40 h with 0.99 μmol L⁻¹ h⁻¹. Fed-batch cultivation lead to Ala as the most excreted amino acid, highest in 5% with up to 2.67 μmol L⁻¹ h⁻¹. It was also noticeable that in 100% Asn was produced 10-fold higher in comparison with the other NH₄⁺ concentrations with up to 0.79 μmol L⁻¹ h⁻¹.

Examining the total amount of excreted amino acids, closed batch shows an increasing excretion of amino acids over time with the highest in later timepoints with 14.67 to 18.44 μmol L-1. Fed-batch shows in general a higher total amount of AA excretion, with highest value of 156.08 μmol L⁻¹ (FIG. 5). On the contrary it is to note, that during closed batch experiments showed a higher production rate [μmol L⁻¹ h⁻¹] than fed-batch experiments. The presence of too much NH₄⁺in the media seems to serve as an inhibitor for amino acid production, as 100% experiments showed slightly lower values (FIG. 5). A comparison of the total uptake of NH₄⁺with the total amino acids excretion rate indicated that amino acid excretion rate increased with increasing AUR. Furthermore, during depletion of NH₄⁺, the concentration of AA did not increase (FIG. 5).

### CONCLUSIONS

In the context of “power to gas” technology, biological methanation with CO₂ originating from renewable sources and in combination with N₂ fixation, the production of amino acids by methanogens is of high economic interest. To our knowledge there is no study yet that examined a combined CO₂/N₂ fixation bioprocess.

A switch of cultivation mode, closed or fed-batch, could alter amino acid excretion rates and concentration, e.g. in the case of Glu from the highest fed-batch value of 2.74 mg L⁻¹ to 44.83 mg L⁻¹ in closed batch (Table 3).

Surprisingly, a variety of amino acids turned out to be actively excreted, with the highest total amount of up to 7.5 μmol L⁻¹ h⁻¹ in early time points (FIG. 5).

These results underscore that a methanogenic microorganism is well suited for production of amino acids in a biotechnological context, even under N₂-fixing conditions.

### Example 2: Amino Acid Production and Active Secretion by Methanothermobacter Marburgensis in Continuous Culture

Continuous culture of M. marburgensis for amino acid production was successfully established.

Experiments were carried out with M. marburgensis in 2 L bioreactors (Eppendorf AG, Hamburg, Germany) and in a 15 L bioreactor (Biostat C+, Sartorius Stedim Biotech AG, Göttingen, Germany).

For fermentation, original MM medium as described in Example 1 above was used.

The same medium was used as feed medium for continuous cultivation mode.

To ensure anaerobic conditions inside the reaction vessel, the whole system was flushed with an H₂/CO₂, N₂ or H₂/CO₂/N₂ mixture for 10 minutes prior to inoculation.

Cultivation was performed at 65° C. and a stirrer speed of 100 to 1200 rpm (DASGIP parallel bioreactor system, Eppendorf AG, Hamburg, Germany) and from 100 to 1500 rpm (Biostat C+, Sartorius Stedim Biotech AG, Göttingen, Germany). The pH was measured by a pH probe (Mettler Toledo GmbH, Vienna, Austria or Hamilton Bonaduz A G, Bonaduz, Switzerland) and kept constant at a value of 7.

The oxidation reduction potential (ORP) was measured by a redox probe (Mettler Toledo GmbH, Vienna, Austria). A 0.5 mol/L Na₂S. 9H₂O solution was used as sulphur source and fed constantly to the bioreactor at e.g. 0.2 mL/h to 1.32 mL/h. The MM medium was applied using an analog peristaltic pump. The MM medium feed flow rate, the sodium Na₂S·9H₂O feed rate and the titration was recorded gravimetrically or adjusted by pump speed. The bioreactor volume was kept constant by withdrawing culture suspension over an immersion pipe using a peristaltic pump controlled on a fixed bioreactor weight, or by using a pipe at a fixed height as a level control system. The withdrawn suspension was collected in a harvest bottle and its volume recorded gravimetrically. All solutions were made anaerobic by flushing with N₂, H₂/CO₂, Or H₂/CO₂/N₂. To maintain anaerobic conditions, all bottles were pressurized with N₂. Pure H₂/CO₂ (4:1) was used as substrate for M. marburgensis. CO₂ gas flow was controlled via the MX4/4 unit (Eppendorf AG, Hamburg, Germany). H₂ gas flow was controlled via the C100 L Unit (Sierra Instruments, Monterey, USA).

About thirty different runs of continuous cultures of M. marburgensis were performed under anaerobic conditions. The volume of a run ranged from 1.6 L to 10.29 L. Dilution rate D was varied between runs, in particular with values for D from 0.0125 h⁻¹ to 0.05 h⁻¹. Volume gas per volume liquid per minute (vvm) was also varied between runs, e.g. from 0.125 to 0.5. Agitation (rpm) was also varied between runs, for instance from 375 to 1500. As a sulfur source, 0.5 mol/L Na₂S was supplied from e.g. 0.2 mL/h to 1.32 mL/h. Typically, the ammonium concentration was kept between 15 mmol/L and 35 mmol/L.

Importantly, volumetric amino acid production rate and specific amino acid production rate ranged from about 25 to about 75 μmol L⁻¹ h⁻¹ and from about 50 to about 2000 μmol h⁻¹g⁻¹, respectively (total over all amino acids). The production and secretion into the culture supernatant of the following amino acids (in combination) was typically observed: Asp, Glu, Asn, Ser, His, Gln, Gly, Thr, Arg, Ala, Tyr, Val, Met, norvaline (Nva), Trp, Ile, Phe, Leu, Lys. Individual amino acid production rates were observed up to about 40 μmol L⁻¹ h⁻¹ (volumetric) and up to about 900 μmol h⁻¹g⁻¹ (specific per biomass). Cys and Pro were not detected due to analytic constraints, but are expected to be produced and secreted as well.

In conclusion, reliable production of amino acids was observed in continuous culture. The secretion of these amino acids to the supernatant is especially remarkable, as it simplifies downstream steps (e.g. no cell lysis required for harvesting the product). Also surprising was the observed production of Nva, which has not been observed before in methanogenic archaea (let alone in Methanobacteriales).

### Example 3: Amino Acid Production and Active Secretion in Further Methanogenic Archaea

Production of amino acids, including canonical amino acids and Nva, and their active secretion were observed in methanogenic archaea other than M. marburgensis, namely in Methanocaldococcus jannaschii, Methanococcus igneus and Methanocaldococcus villosus. These methanogenic microorganisms were incubated under closed batch conditions similar to the conditions disclosed in Example 1, but at their respective preferred temperatures in 282-based medium (see also Mauerhofer et al, 2021). While for instance Glu production was more pronounced under these conditions, Nva production was also clearly observed for each of Methanocaldococcus jannaschii, Methanococcus igneus and Methanocaldococcus villosus (volumetric Nva production rates reached beyond 1.0 μmol L⁻¹ h⁻¹, specific Nva production rates beyond 10 μmol g⁻¹ h⁻¹). Production of Nva has not been observed before in methanogenic archaea (let alone in Methanococcales).

In summary, the production and active secretion into the culture supernatant of the following amino acids (in combination) was observed for methanogenic microorganisms: Asp, Glu, Asn, Ser, His, Gln, Gly, Thr, Arg, Ala, Tyr, Val, Met, Nva, Trp, Ile, Phe, Leu and Lys.

### Example 4: Amino Acid Production and Active Secretion by Methanothermobacter Marburgensis in Continuous Culture (Further Experiments)

Experiments were carried out with M. marburgensis in 2.2 L bioreactors (Eppendorf AG, Hamburg, Germany) and in a 15 L bioreactor (Biostat C+, Sartorius Stedim Biotech AG, Göttingen, Germany).

For fermentation, original MM medium as described in Example 1 above was used.

The same medium was used as feed medium for continuous cultivation mode.

To ensure anaerobic conditions inside the reaction vessel, the whole system was flushed with an H₂/CO₂, N₂ or H₂/CO₂/N₂ mixture for 10 minutes prior to inoculation.

Cultivation was performed at 65° C. and a stirrer speed of 375 to 1500 rpm (DASGIP parallel bioreactor system, Eppendorf AG, Hamburg, Germany) and from 375 to 1500 rpm (Biostat C+, Sartorius Stedim Biotech AG, Göttingen, Germany). The pH was measured by a pH probe (Mettler Toledo GmbH, Vienna, Austria or Hamilton Bonaduz AG, Bonaduz, Switzerland) and kept constant at a value of 7.

The oxidation reduction potential (ORP) was measured by a redox probe (Mettler Toledo GmbH, Vienna, Austria). A 0.5 mol/L Na₂S·9H₂O solution was used as sulphur source and fed constantly to the bioreactor at e.g. 0.05 mL/h to 1.32 mL/h. The MM medium was applied using an analog peristaltic pump. The MM medium feed flow rate, the sodium Na₂S·9H₂O feed rate and the titration was adjusted by pump speed. The bioreactor volume was kept constant. The withdrawn suspension was collected in a harvest bottle. All solutions were made anaerobic by flushing with N₂, H₂/CO₂, or H₂/CO₂/N₂. To maintain anaerobic conditions, all bottles were pressurized with N₂. Pure H₂/CO₂ (4:1) was used as substrate for M. marburgensis. CO₂ gas flow was controlled via the MX4/4 unit (Eppendorf AG, Hamburg, Germany). H₂ gas flow was controlled via the C100 L Unit (Sierra Instruments, Monterey, USA).

More than 100 different runs of continuous cultures of M. marburgensis were performed under anaerobic conditions. The volume of a run ranged from 1.6 L to 10.29 L. Dilution rate D was varied between runs, in particular with values for D from 0.00625 h⁻¹ to 0.05 h⁻¹, such as 0.025 h⁻¹. Volume gas per volume liquid per minute (vvm) was also varied between runs, e.g. from 0.125 to 0.5. Agitation (rpm) was also varied between runs, for instance from 375 to 1500. As a sulfur source, 0.5 mol/L Na₂S was supplied from e.g. 0.05 mL/h to 1.32 mL/h. Typically, the ammonium concentration was kept between 0.21 mmol/L and 41.63 mmol/L.

Importantly, volumetric amino acid production rate and specific amino acid production rate ranged from about 5 to about 250 μmol L⁻¹ h⁻¹ and from about 10 to about 150 μmol h⁻¹g⁻¹, respectively (total over all amino acids). The production and secretion into the culture supernatant of the following amino acids (in combination) was typically observed: Asp, Glu, Ser, His, Gly, Thr, Arg, Ala, Tyr, Val, Met, norvaline (Nva), Trp, Ile, Phe, Leu, norleucine (Nle), Lys. Individual amino acid production rates were observed up to about 130 μmol L⁻¹ h⁻¹ (volumetric) and up to about 70 μmol h⁻¹g⁻¹ (specific per biomass). Cys and Pro were not detected due to analytic constraints, but are expected to be produced and secreted as well.

In conclusion, reliable production of amino acids was observed in continuous culture. The secretion of these amino acids to the supernatant is especially remarkable, as it simplifies downstream steps (e.g. no cell lysis required for harvesting the product). Also surprising was the observed production of Nva and Nle, which has not been observed before in methanogenic archaea (let alone in Methanobacteriales).

### Example 5: Amino Acid Production and Active Secretion in Further Methanogenic Archaea

Production of amino acids, including canonical amino acids, norvaline, ornithine and homoserine, and their active secretion into the culture supernatant were observed in methanogenic archaea other than M. marburgensis, namely in Methanothermobacter thermautotrophicus and a Methanothermococcus sp. These methanogenic microorganisms were incubated under closed batch conditions similar to the conditions disclosed in Example 1, but at their respective preferred temperatures in MM medium (see Example 1; for Methanothermococcus culture, 30 g/L NaCl was added to the medium) and at various ammonium concentrations. While for instance Ala and Glu production was more pronounced under these conditions, Nva production was also clearly observed for each. Production of Nva has not been observed before in methanogenic archaea (let alone in Methanobacteriales or Methanococcales).

In summary, the production and active secretion into the culture supernatant of the following amino acids (in combination) was observed for methanogenic microorganisms: Ala, Asp, Asn, Glu, Gly, His, homoserine, Ile, Leu/Nle, Lys, Nva, ornithine, Phe, Pro, Ser, Thr, Try, Tyr and Val. (The detection method used in this example was not able to distinguish between Leu and Nle.)

### Non-Patent References

Abdel Azim, A., Rittmann, S. K. -M. R., Fino, D., Bochmann, G., 2018. The physiological effect of heavy metals and volatile fatty acids on Methanococcus maripaludis S2. Biotechnol Biofuels 11, 301.
Abdel Azim, Annalisa, Christian Pruckner, Philipp Kolar, Ruth Sophie Taubner, Debora Fino, Guido Saracco, Filipa L. Sousa, and Simon K. M. R. Rittmann. 2017. “The Physiology of Trace Elements in Biological Methane Production.” Bioresource Technology 241:775-86.
Becker J and Wittmann C. Systems and synthetic metabolic engineering for amino acid production—the heartbeat of industrial strain development. Curr Opin Biotechnol. 2012 October; 23 (5): 718-26.
Belay N, Sparling R and Daniels L. 1984. “Dinitrogen Fixation by a Thermophilic Methanogenic Bacterium.” Nature 312 (5991): 286-288.
Bellack, Annett, Harald Huber, Reinhard Rachel, Gerhard Wanner, and Reinhard Wirth. 2011. “Methanocaldococcus Villosus Sp. Nov., a Heavily Flagellated Archaeon That Adheres to Surfaces and Forms Cell-Cell Contacts.”
Bernacchi, Sébastien, et al. “Experimental methods for screening parameters influencing the growth to product yield (Y (x/CH₄)) of a biological methane production (BMP) process performed with Methanothermobacter marburgensis.” AIMS Bioengineering 1.2 (2014): 72-87.
Biermann, Michael, et al. “Simultaneous analysis of the non-canonical amino acids norleucine and norvaline in biopharmaceutical-related fermentation processes by a new ultra-high performance liquid chromatography approach.” Amino Acids 44.4 (2013): 1225-1231.
Blank, C. E., Peter S. Kessler, and John A. Leigh. 1995. “Genetics in Methanogens: Transposon Insertion Mutagenesis of a Methanococcus Maripaludis NifH Gene.” Journal of Bacteriology 177 (20): 5773-77.
Blotevogel, Karl Heinz and Ulrich Fischer. 1985. “Isolation and Characterization of a New Thermophilic and Autotrophic Methane Producing Bacterium: Methanobacterium Thermoaggregans Spec. Nov.” Archives of Microbiology 142 (3): 218-22.
Bult, Carol J., et al. “Complete genome sequence of the methanogenic archaeon, Methanococcus jannaschii.” Science 273.5278 (1996): 1058-1073.
Dhamad, Ahmed E., and Daniel J. Lessner. “A CRISPRi-dCas9 system for archaea and its use to examine gene function during nitrogen fixation by Methanosarcina acetivorans.” Applied and environmental microbiology 86.21 (2020): e01402-20.
Dridi, Bédis, et al. “Tungsten-enhanced growth of Methanosphaera stadtmanae.” BMC Research Notes 5 (2012): 1-4.
Fardeau, M-L., J-P. Peillex, and J-P. Belaich. “Energetics of the growth of Methanobacterium thermoautotrophicum and Methanococcus thermolithotrophicus on ammonium chloride and dinitrogen.” Archives of microbiology 148.2 (1987): 128-131.
Fernandez L, Bertilsson S, Peura S 2019 Non-cyanobacterial diazotrophs dominate nitrogen-fixing communities in permafrost thaw ponds, Limnol. Oceanogr. 65, 2020, S180-S193, Association for the Sciences of Limnology and Oceanography.
Hoffarth, Marc Philippe, Timo Broeker, and Jan Schneider. “Effect of N₂ on biological methanation in a continuous stirred-tank reactor with methanothermobacter marburgensis.” Fermentation 5.3 (2019): 56.
Hu Y and Ribbe M W. 2012. “Nitrogenase Assembly.” Biochimica et Biophysica Acta 1827:1112-22.
Jones, J. B., and Stadtman. “Methanococcus vannielii: culture and effects of selenium and tungsten on growth.” Journal of bacteriology 130.3 (1977): 1404-1406.
Jones, W. Jack, M. J. B. Paynter, and R. Gupta. 1983. Characterization of Methanococcus Maripaludis Sp. Nov., a New Methanogen Isolated from Salt Marsh Sediment. Vol. 135.
Kandeler, Ellen. 1988. “Short-Term Assay of Soil Urease Activity Using Colorimetric Determination of Ammonium SoilCare View Project Biodiversity Exploratories View Project.” Biology and Fertility of Soils 6 (1): 68-72.
Kengen, Serve W M, and Alfons J M Stams. “Formation of L-alanine as a reduced end product in carbohydrate fermentation by the hyperthermophilic archaeon Pyrococcus furiosus.” Archives of Microbiology 161 (1994): 168-175.
Kessler, Peter S, Jennifer McLarnan, and John A. Leigh. “Nitrogenase phylogeny and the molybdenum dependence of nitrogen fixation in Methanococcus maripaludis.” Journal of bacteriology 179.2 (1997): 541-543.
Kisumi, Masahiko, Masaki Sugiura, and Ichiro Chibata. “Biosynthesis of norvaline, norleucine, and homoisoleucine in Serratia marcescens.” The Journal of Biochemistry 80.2 (1976): 333-339.
Kurth J M, Op den Camp H J M, Welte C U, 2020. Several ways one goal-methanogenesis from unconventional substrates. Appl Microbiol Biotechnol 104, 6839-6854.
Leigh, John A. 2000. “Nitrogen Fixation In Methanogens: The Archaeal Perspective.” 2:125-31.
Liu, Hui, et al. “Effects of different amino acids and their configurations on methane yield and biotransformation of intermediate metabolites during anaerobic digestion.” Journal of Environmental Management 296 (2021): 113152.
Lobo, Anthony L., and Stephen H. Zinder. “Diazotrophy and nitrogenase activity in the archaebacterium Methanosarcina barkeri 227.” Applied and environmental microbiology 54.7 (1988): 1656-1661.
Lyu, Z, Shao N, Akinyemi T, Whitman W B, 2018. Methanogenesis. Current Biology 28, R727-R732.
Mand, Thomas D., and William W. Metcalf. “Energy conservation and hydrogenase function in methanogenic archaea, in particular the genus Methanosarcina.” Microbiology and Molecular Biology Reviews 83.4 (2019): e00020-19.
Mauerhofer, L M., Zwirtmayr, S, Pappenreiter, P. et al. Hyperthermophilic methanogenic archaea act as high-pressure CH₄ cell factories. Commun Biol 4, 289 (2021).
Mauerhofer, L.-M., Reischl, B., Schmider, T., Schupp, B., Nagy, K., Pappenreiter, P., Zwirtmayr, S., Schuster, B., Bernacchi, S., Seifert, A. H., Paulik, C., Rittmann, S. K.-M. R., 2018. Physiology and methane productivity of Methanobacterium thermaggregans. Appl Microbiol Biotechnol 102, 7643-7656.
Mayumi D, Hanako M, Hideyuki T, Kyosuke Y, Hideyoshi Y, Yuichiro S, Yoichi K, and Susumu S. 2016. “Methane Production from Coal by a Single Methanogen.” Science 354 (6309): 222-25.
Murray P A and Zinder S H. 1984. “Nitrogen Fixation by a Methanogenic Archaebacterium.” Nature 312 (5991): 284-86.
Nayak, Dipti D., and William W. Metcalf. “Cas9-mediated genome editing in the methanogenic archaeon Methanosarcina acetivorans.” Proceedings of the National Academy of Sciences 114.11 (2017): 2976-2981.
Pappenreiter, P. A., Zwirtmayr, S., Mauerhofer, L.-M., Rittmann, S. K.-M. R., Paulik, C., 2019. Development of a simultaneous bioreactor system for characterization of gas production kinetics of methanogenic archaea at high pressure. Engineering in Life Sciences 19, 537-544.
Pfeifer, Kevin, et al. “Archaea biotechnology.” Biotechnology Advances 47 (2021): 107668.
Polis, Baruh, et al. “L-Norvaline, a new therapeutic agent against Alzheimer's disease.” Neural regeneration research 14.9 (2019): 1562.
Porat, Iris, et al. “Two biosynthetic pathways for aromatic amino acids in the archaeon Methanococcus maripaludis.” Journal of bacteriology 186.15 (2004): 4940-4950.
Raymond J, Siefert J L, Staples C R, and Blankenship R E. 2004. “The Natural History of Nitrogen Fixation.” Molecular Biology and Evolution 21 (3): 541-54.
Rinke, Christian, et al. “A standardized archaeal taxonomy for the Genome Taxonomy Database.” Nature Microbiology 6.7 (2021): 946-959.
Rittmann, S., A. Seifert, and Christoph Herwig. “Quantitative analysis of media dilution rate effects on Methanothermobacter marburgensis grown in continuous culture on H₂ and CO₂·” Biomass and Bioenergy 36 (2012): 293-301.
Rittmann, S. K.-M. R., 2015. A Critical Assessment of Microbiological Biogas to Biomethane Upgrading Systems. Adv. Biochem. Eng. Biotechnol. 151, 117-135.
Rittmann, S. K.-M. R., Seifert, A. H., Bernacchi, S., 2018. Kinetics, multivariate statistical modelling, and physiology of CO₂-based biological methane production. Applied Energy 216, 751-760.
Rittmann, Simon K-M R, et al. “Archaea in der Biotechnologie.” BIOspektrum 27.1 (2021): 96-98.
Sarmiento, Felipe B., John A. Leigh, and William B. Whitman. “Genetic systems for hydrogenotrophic methanogens.” Methods in enzymology. Vol. 494. Academic Press, 2011. 43-73.
Schönheit P, Johanna Moll, and Rudolf K. Thauer. 1980. “Growth Parameters (Ks, Mmax, Ys) of Methanobacterium Thermoautotrophicum.” Archives of Microbiology 127 (1): 59-65
Schönheit, Peter, and Rudolf K. Thauer. “L-Alanine, a product of cell wall synthesis in Methanobacterium thermoautotrophicum.” FEMS Microbiology Letters 9.2 (1980): 77-80.
Sment & Konisky. “Excretion of amino acids by 1, 2, 4-triazole-3-alanine-resistant mutants of Methanococcus voltae.” Applied and environmental microbiology 55.5 (1989): 1295-1297.
Takai, Ken, Akira Inoue, and Koki Horikoshi. 2002. “Methanothermococcus Okinawensis Sp. Nov., a Thermophilic, Methane-Producing Archaeon Isolated from a Western Pacific Deep-Sea Hydrothermal Vent System.” International Journal of Systematic and Evolutionary Microbiology 52 (4): 1089-95.
Taubner, Ruth Sophie and Simon K. M. R. Rittmann. 2016. “Method for Indirect Quantification of CH4 production via H₂O Production Using Hydrogenotrophic Methanogens.” Frontiers in Microbiology 7 (APR): 1-11.
Taubner, R.-S., et al, 2018. Biological methane production under putative Enceladus-like conditions. Nature Communications 9, 748.
Taubner, Ruth-Sophie, et al. “Membrane lipid composition and amino acid excretion patterns of Methanothermococcus okinawensis grown in the presence of inhibitors detected in the Enceladian plume.” Life 9.4 (2019): 85.
Thamdrup B, New Pathways and Processes in the Global Nitrogen Cycle, 2012, Annual Review of Ecology, Evolution, and Systematics, 407-428, 43, 1.
Wasserfallen A, et al. 2000. “Phylogenetic Analysis of 18 Thermophilic Methanobacterium Isolates Supports the Proposals to Create a New Genus, Methanothermobacter Gen. Nov., and to Reclassify Several Isolates in Three Species, Methanothermobacter Thermautotrophicus Comb. Nov., Methano.” International Journal of Systematic and Evolutionary Microbiology 50:43-53.
Whitman, W. B., Es Ankwanda, and R. S. Wolfe. “Nutrition and carbon metabolism of Methanococcus voltae.” Journal of Bacteriology 149.3 (1982): 852-863.
Whitman, William B., et al. “Isolation and characterization of 22 mesophilic methanococci.” Systematic and applied microbiology 7.2-3 (1986): 235-240.
Zeikus, J. G. “The biology of methanogenic bacteria.” Bacteriological reviews 41.2 (1977): 514-541.

1-15. (canceled)

16. A method of producing amino acids by fermentation in a bioreactor, wherein the bioreactor comprises methanogenic microorganisms of the genus Methanothermobacter in a fermentation broth, wherein the fermentation broth comprises ammonium at a concentration of 0.1 mmol/L to 200 mmol/L, the method comprising at least the steps of:
feeding a gaseous carbon source comprising carbon dioxide and/or carbon monoxide, a nitrogen source and a sulfur source to the bioreactor under conditions such that the methanogenic microorganisms produce the amino acids; and
harvesting at least a portion of the amino acids from the supernatant of the fermentation broth, wherein the harvesting comprises removing a liquid stream from the bioreactor, wherein the liquid stream comprises fermentation broth, methanogenic microorganisms, and produced amino acids in the supernatant, separating the methanogenic microorganisms from the liquid stream and recycling the methanogenic microorganisms back to the bioreactor;
wherein the total amino acid production rate per biomass is at least 0.1 μmol g⁻¹ h⁻¹.

17. The method of claim 16, wherein the supernatant has a protein content of less than 1000 μg/mL.

18. The method of claim 16, wherein at least 50% of the methanogenic microorganisms remain viable and/or intact prior to and during the harvesting.

19. The method of claim 16, wherein said portion comprises at least two of the 20 canonical amino acids.

20. The method of claim 16, wherein the total amino acid concentration in the supernatant of the fermentation broth is at least 1 μmol/L.

21. The method of claim 16, wherein the methanogenic microorganisms comprise Methanothermobacter marburgensis.

22. The method of claim 16, wherein the total amino acid production rate per volume of fermentation broth is at least 0.01 μmol L⁻¹ h⁻¹.

23. The method of claim 16, wherein the method is a continuous process, a fed-batch process, a batch process, a closed batch process, a repetitive batch process, a repetitive fed-batch process or a repetitive closed batch process.

24. The method of claim 16, where the nitrogen source comprises nitrogen gas; wherein at least 50% of all nitrogen atoms of all nitrogen sources fed to the bioreactor are fed to the bioreactor in the form of nitrogen gas.

25. The method of claim 16, wherein at least 50% of all carbon atoms of all carbon sources fed to the bioreactor are fed to the bioreactor in the form of carbon dioxide gas and/or carbon monoxide gas.

26. The method of claim 16, wherein the sulfur source is fed to the bioreactor, wherein the sulfur source comprises cysteine and/or sulfide, especially wherein the fermentation broth comprises sulfide at a concentration of 0.001-150 mg/L.

27. The method of claim 16, wherein hydrogen gas, acetate, a methyl compound selected from methylamines, methyl sulfides and methanol, any other alcohol, a secondary alcohol such as 2-propanol or 2-butanol, a methoxylated aromatic compound and/or formate are fed to the bioreactor; wherein hydrogen gas, acetate, methanol or combinations thereof are fed to the bioreactor.

28. A use of methanogenic microorganisms of the genus Methanothermobacter for producing amino acids, wherein the amino acids are harvested from the supernatant of the fermentation broth, wherein the harvesting comprises removing a liquid stream from the bioreactor, wherein the liquid stream comprises fermentation broth, methanogenic microorganisms, and produced amino acids in the supernatant, separating the methanogenic microorganisms from the liquid stream and recycling the methanogenic microorganisms back to the bioreactor;
wherein the total amino acid production rate per biomass is at least 0.1 μmol g⁻¹ h⁻¹¹;
wherein the fermentation broth comprises ammonium at a concentration of 0.1 mmol/L to 200 mmol/L.
